(19) United States
(12) Patent Application Publication
(10) Pub. No.: US 2023/0050945 A1
(43) Pub. Date: Feb. 16, 2023

(54) IMAGE PROCESSING SYSTEM, ENDOSCOPE SYSTEM, AND IMAGE PROCESSING METHOD

(71) Applicant: OLYMPUS CORPORATION, Tokyo (JP)

(72) Inventor: Yuri NAKAUE, Sagamihara-shi (JP)

(73) Assignee: OLYMPUS CORPORATION, Tokyo (JP)

(21) Appl. No.: 17/974,626

(22) Filed: Oct. 27, 2022

Related U.S. Application Data
(63) Continuation of application No. PCT/JP2020/018964, filed on May 12, 2020.

(51) Int. Cl. G06T 7/00; G06V 10/143; G06V 10/25; G06V 10/70

(52) U.S. Cl. CPC G06T 7/0012; G06V 10/143; G06V 10/25; G06V 10/70; G06T 2207/10068; G06T 2207/10152

(57) ABSTRACT

An image processing system includes a processor, the processor performing processing, based on association information of an association between a biological image captured under a first imaging condition and a biological image captured under a second imaging condition, of outputting a prediction image corresponding to an image in which an object captured in an input image is to be captured under the second imaging condition. The association information is indicative of a trained model obtained through machine learning of a relationship between a first training image captured under the first imaging condition and a second training image captured under the second imaging condition. The processor is capable of outputting a plurality of different kinds of prediction images based on a plurality of trained models and the input image, and performs processing, based on a given condition, of selecting the prediction image to be output among a plurality of prediction images.

### CROSS REFERENCE TO RELATED APPLICATION

This application is a continuation of International Patent Application No. PCT/JP2020/018964, having an international filing date of May 12, 2020, which designated the United States, the entirety of which is incorporated herein by reference.

### BACKGROUND

Conventionally, a method of capturing an image of a living body under different imaging conditions is known. For example, imaging with white light, as well as imaging with special light and imaging with pigments dispersion on an object have been performed. By performing special light observation or pigments dispersion observation, it is possible to highlight blood vessels, unevenness, etc., and thus support diagnostic imaging by a physician.

For example, Japanese Unexamined Patent Application Publication No. 2012-70935 discloses a method wherein both white illumination light and purple narrow band light are emitted to one frame, the method selectively reducing intensity of a specific color component to display an image having color tones similar to those by white light observation. In addition, Japanese Unexamined Patent Application Publication No. 2016-2133 discloses a method for obtaining an image in which dye is substantially not visually recognized, by using dye invalid illumination light in a pigments dispersed state.

Furthermore, Japanese Unexamined Patent Application Publication No. 2000-115553 discloses a spectral estimation technique that estimates signal components of a predetermined wavelength band based on a white light image and an optical spectrum of a living body as an object.

### SUMMARY

In accordance with one of some aspect, there is provided an image processing system comprising a processor including hardware, the processor being configured to: obtain, as an input image, a biological image captured under a first imaging condition; and perform processing, based on association information of an association between the biological image captured under the first imaging condition and the biological image captured under a second imaging condition that differs from the first imaging condition, of outputting a prediction image corresponding to an image in which an object captured in the input image is to be captured under the second imaging condition, wherein the association information is indicative of a trained model obtained through machine learning of a relationship between a first training image captured under the first imaging condition and a second training image captured under the second imaging condition, the processor is capable of outputting, based on a plurality of the trained models and the input image, a plurality of different kinds of the prediction images, and the processor performs processing, based on a given condition, of selecting the prediction image to be output among a plurality of the prediction images.

In accordance with one of some aspect, there is provided an endoscope system comprising: an illumination device irradiating an object with illumination light; an imaging device outputting a biological image in which the object is captured; and a processor including hardware, wherein the processor is configured to: obtain, as an input image, the biological image captured under a first imaging condition and perform processing, based on association information of an association between the biological image captured under the first imaging condition and the biological image captured under a second imaging condition that differs from the first imaging condition, of outputting a prediction image corresponding to an image in which the object captured in the input image is to be captured under the second imaging condition, the association information is indicative of a trained model obtained through machine learning of a relationship between a first training image captured under the first imaging condition and a second training image captured under the second imaging condition, the processor is capable of outputting, based on a plurality of the trained models and the input image, a plurality of different kinds of the prediction images, and the processor performs processing, based on a given condition, of selecting the prediction image to be output among a plurality of the prediction images.

In accordance with one of some aspect, there is provided an image processing method comprising: obtaining, as an input image, a biological image captured under a first imaging condition; obtaining association information of an association between the biological image captured under the first imaging condition and the biological image captured under a second imaging condition that differs from the first imaging condition; and outputting, based on the input image and the association information, a prediction image corresponding to an image in which an object captured in the input image is to be captured under the second imaging condition, wherein the association information is indicative of a trained model obtained through machine learning of a relationship between a first training image captured under the first imaging condition and a second training image captured under the second imaging condition, and the method is capable of outputting, based on a plurality of the trained models and the input image, a plurality of different kinds of the prediction images, and performs processing, based on a given condition, of selecting the prediction image to be output among a plurality of the prediction images.

### BRIEF DESCRIPTION OF THE DRAWINGS

FIG. 1 illustrates an example configuration of a system including an image processing system.

FIG. 2 illustrates an example configuration of the image processing system.

FIG. 3 is an external view of an endoscope system.

FIG. 4 illustrates an example configuration of the endoscope system.

FIG. 5A is a diagram illustrating a wavelength band of illumination light that constitutes white light, and FIG. 5B is a diagram illustrating a wavelength band of illumination light that constitutes special light.

FIG. 6A illustrates an example of a white light image and FIG. 6B illustrates an example of a pigments dispersed image.

FIG. 7 illustrates an example configuration of a learning device.

FIGS. 8A and 8B illustrate example configurations of a neural network.

FIG. 9 is a diagram illustrating input/output of a trained model.

FIG. 10 is a flowchart illustrating learning processing.

FIG. 11 is a flowchart illustrating processing in the image processing system.

FIGS. 12A to 12C illustrate example screens on which a prediction image is displayed.

FIG. 13 is a diagram illustrating input/output of a plurality of trained models outputting a prediction image.

FIGS. 14A and 14B are diagrams illustrating input/output of a trained model detecting a region of interest.

FIG. 15 is a flowchart illustrating processing of switching modes.

FIGS. 16A and 16B are diagrams illustrating a configuration of an illumination section.

FIGS. 17A and 17B are diagrams illustrating input/output of a trained model outputting a prediction image.

FIG. 18 is a flowchart illustrating processing in the image processing system.

FIG. 19 is a diagram illustrating a relationship between an imaging frame of an image and processing.

FIGS. 20A and 20B illustrate example configurations of a neural network.

FIG. 21 is a diagram illustrating input/output of a trained model outputting a prediction image.

FIG. 22 is a diagram illustrating a relationship between an imaging frame of an image and processing.

FIG. 23 is a diagram illustrating input/output of a trained model outputting a prediction image.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

### 1. First Embodiment

1.1 System Configuration

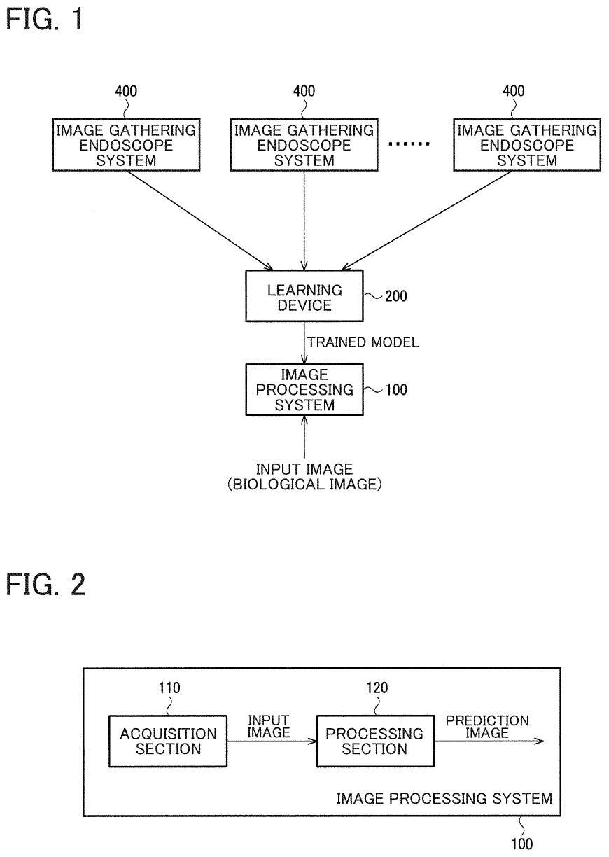

FIG. 1 illustrates an example configuration of a system including an image processing system 100 according to the present embodiment. As illustrated in FIG. 1, the system includes the image processing system 100, a learning device 200, and an image gathering endoscope system 400. However, the system is not limited to the configuration in FIG. 1, and can be implemented in various modifications, such as by omitting some of these components or adding other components. For example, machine learning is not essential to the present embodiment and thus the learning device 200 may be omitted.

The image gathering endoscope system 400 captures a plurality of biological images for generating a trained model. That is, the biological images captured by the image gathering endoscope system 400 are indicative of training data to be used for machine learning. For example, the image gathering endoscope system 400 outputs a first training image in which a given object is captured under a first imaging condition and a second training image in which the same object is captured under a second imaging condition. On the contrary, an endoscope system 300 described later is different in that it captures an image under the first imaging condition, but does not need to capture an image under the second imaging condition.

The learning device 200 obtains a pair of the first training image and the second training image captured by the image gathering endoscope system 400 as the training data to be used for machine learning. The learning device 200 generates a trained model through machine learning based on the training data. Specifically, the trained model is a model that performs inference processing based on deep learning. The learning device 200 transmits the generated trained model to the image processing system 100.

FIG. 2 illustrates a configuration of the image processing system 100. The image processing system 100 includes an acquisition section 110 and a processing section 120. However, the image processing system 100 is not limited to the configuration in FIG. 2, and can be implemented in various modifications, such as by omitting some of these components or adding other components.

The acquisition section 110 obtains, as an input image, a biological image captured under the first imaging condition. The input image is captured by an imaging section of the endoscope system 300, for example. Specifically, the imaging section corresponds to an image sensor 312 described later. Specifically, the acquisition section 110 is an interface for inputting/outputting an image.

The processing section 120 obtains the trained model generated by the learning device 200. For example, the image processing system 100 includes a storage section (not shown) that stores the trained model generated by the learning device 200. The storage section herein is to be a work domain of the processing section 120 or the like, and its function can be implemented by a semiconductor memory, a register, a magnetic storage device or the like. The processing section 120 reads out the trained model from the storage section and operates following instructions from the trained model, thereby performing inference processing based on the input image. For example, the image processing system 100 performs processing, based on the input image in which a given object is captured under the first imaging condition, of outputting a prediction image corresponding to an image in which the object is to be captured under the second imaging condition.

Note that the processing section 120 is configured with the following hardware. The hardware can include at least one of a circuit that processes digital signals and a circuit that processes analog signals. For example, the hardware can be configured with one or more circuit devices mounted on a circuit board, or one or more circuit elements. The one or more circuit devices are, for example, IC (Integrated Circuit), FPGA (field-programmable gate array), or the like. The one or more circuit elements are, for example, a register, a capacitor, or the like.

In addition, the processing section 120 may be implemented by the following processor. The image processing system 100 includes a memory that stores information and a processor that operates based on the information stored in the memory. The memory herein may be the storage section described above or a different memory. The information includes, for example, a program and various kinds of data, etc. The processor includes hardware. As the processor, various processors such as CPU (Central Processing Unit), GPU (Graphics Processing Unit), DSP (Digital Signal Processor) or the like can be used. The memory may be a semiconductor memory such as SRAM (Static Random Access Memory) and DRAM (Dynamic Random Access Memory), a register, a magnetic storage device such as HDD (Hard Disk Drive), or an optical storage device such as an optical disk device. For example, the memory stores computer readable instructions, and the processor executes the instructions to realize functions of the processing section 120 as processing. The functions of the processing section 120 are, for example, a function of each section including a prediction processing section 334, a detection processing section 335, a postprocessing section 336, etc. described later. The instructions herein may be a set of instructions that constitutes a program, or instructions that instruct the hardware circuit of the processor to operate. Further, all or some sections of the processing section 120 can be implemented by cloud computing, and each processing described later can be performed on cloud computing.

Further, the processing section 120 in the present embodiment may be implemented as a module of a program that runs on the processor. For example, the processing section 120 is implemented as an image processing module that obtains a prediction image based on an input image.

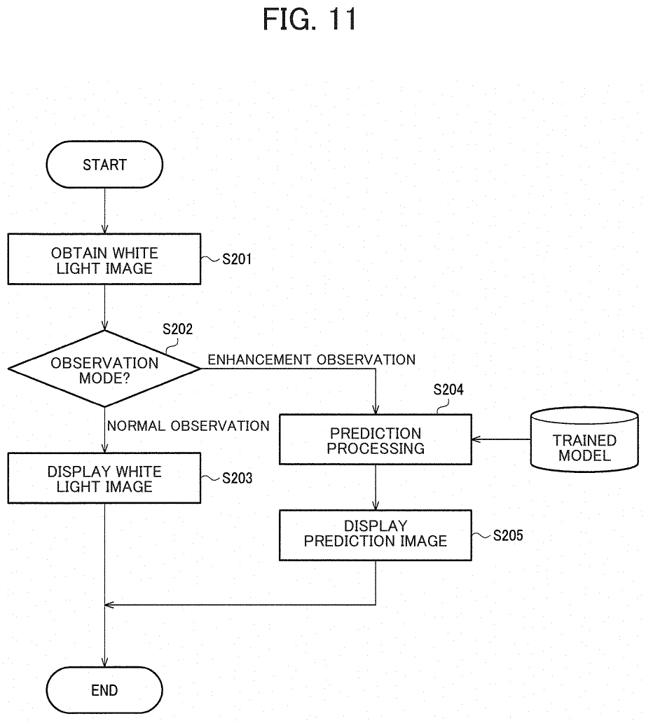

Furthermore, the programs for implementing processing performed by the processing section 120 in the present embodiment can be stored in, for example, an information storage device that is a computer readable medium. The information storage device can be implemented by, for example, an optical disk, a memory card, HDD, or a semiconductor memory. The semiconductor memory is, for example, ROM. The processing section 120 performs various processing in the present embodiment based on the programs stored in the information storage device. That is, the information storage device stores the programs that make the computer function as the processing section 120. The computer is a device equipped with an input device, a processing section, a storage section, and an output section. Specifically, the program according to the present embodiment is a program that makes the computer execute each step described later with reference to FIG. 11 etc.

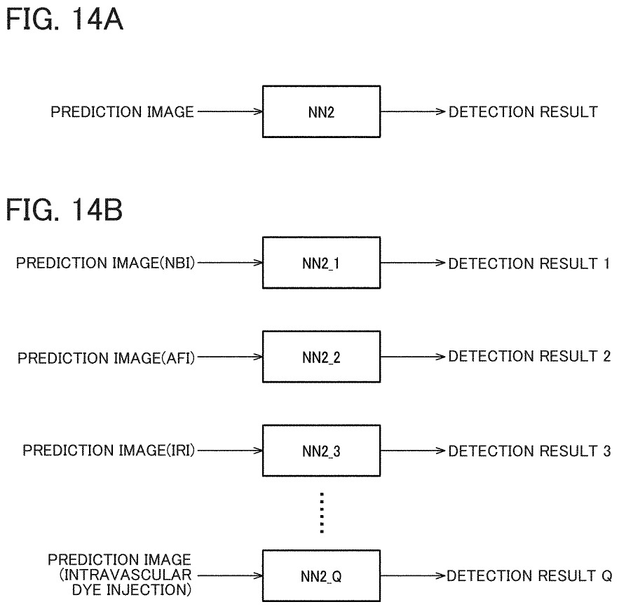

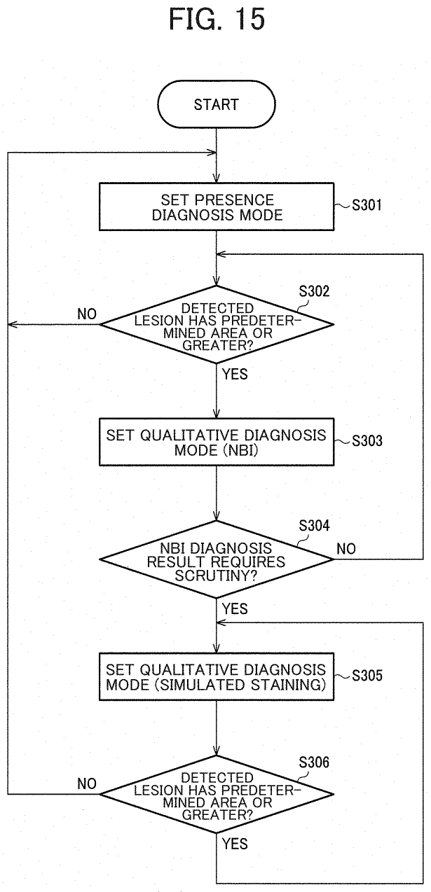

Also as described later with reference to FIGS. 14 and 15, the image processing system 100 in the present embodiment may perform processing of detecting a region of interest from a prediction image. For example, the learning device 200 may have an interface that receives annotation results from a user. The annotation results herein are information to be input by a user, for example, information specifying a position, a shape, a type, etc. of a region of interest. The learning device 200 outputs a trained model for detecting a region of interest through machine learning using, as the training data, the second training image and the annotation results for the second training image. The image processing system 100 may also perform processing of detecting a region of interest from an input image. In this case, the learning device 200 outputs the trained model for detecting the region of interest through machine learning using, as the training data, the first training image and the annotation results for the first training image.

In the system illustrated in FIG. 1, the biological images obtained in the image gathering endoscope system 400 are directly transmitted to the learning device 200, but the method of the present embodiment is not limited thereto. For example, the system including the image processing system 100 may include a server system (not shown).

The server system may be a server provided on a private network such as an intranet, or a server provided on a public communication network such as the Internet. The server system collects a training image, which is a biological image, from the image gathering endoscope system 400. The learning device 200 may obtain the training image from the server system and generate a trained model based on the training image.

The server system may also obtain the trained model generated by the learning device 200. The image processing system 100 obtains the trained model from the server system and performs processing, based on the trained model, of outputting a prediction image and detecting a region of interest. Using the server system in this manner enables efficient accumulation and use of the training image and the trained model.

Further, the learning device 200 and the image processing system 100 may be configured integrally with each other. In this case, the image processing system 100 performs both processing of generating a trained model through machine learning and inference processing based on the trained model.

As described above, FIG. 1 illustrates one example of the system configuration, and various modifications can be made to the configuration of the system including the image processing system 100.

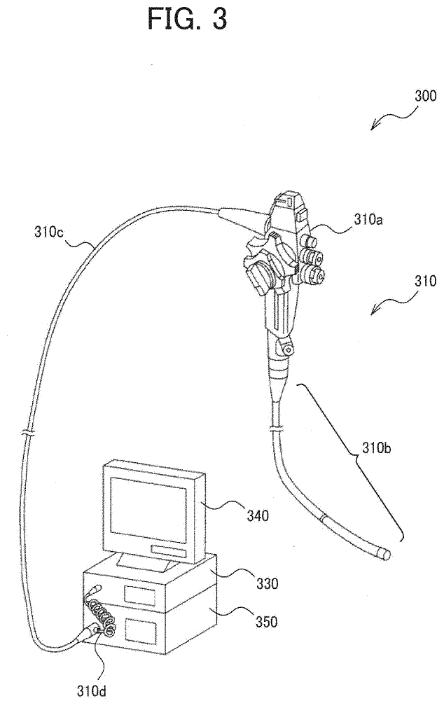

FIG. 3 illustrates a configuration of the endoscope system 300 including the image processing system 100. The endoscope system 300 includes a scope section 310, a processing device 330, a display section 340, and a light source device 350. For example, the image processing system 100 is included in the processing device 330. A physician uses the endoscope system 300 to perform endoscopy for a patient. However, the configuration of the endoscope system 300 is not limited to the one in FIG. 3, and can be implemented in various modifications, such as by omitting some of the components or adding other components. Also illustrated below is a flexible scope used for diagnosis of digestive tracts or the like, but the scope section 310 according to the present embodiment may be a rigid scope used for laparoscopic surgery or the like.

Further, FIG. 3 illustrates one example in which the processing device 330 is a single device connected to the scope section 310 via a connector 310d, but not limited thereto. For example, some or all of the configurations of the processing device 330 may be constructed through any other information processing device such as PC (Personal Computer) or a server system that can be connected via a network. For example, the processing device 330 may be implemented by cloud computing. The network herein may be a private network such as an intranet or a public communication network such as the Internet. The network may also be wired or wireless. That is, the image processing system 100 in the present embodiment is not limited to the configuration included in equipment connected via the scope section 310 and the connector 310d; some or all of the functions thereof may be implemented by other equipment such as PC, or may be implemented by cloud computing.

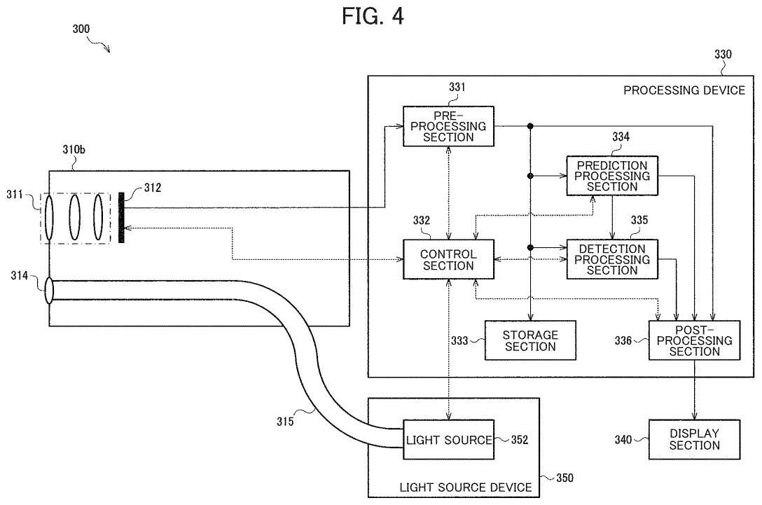

The scope section 310 has an operation section 310a, a flexible insertion section 310b, and a universal cable 310c including a signal line or the like. The scope section 310 is a tubular insertion device with the tubular insertion section 310b to be inserted into a body cavity. The connector 310d is provided at the leading end of the universal cable 310c. The scope section 310 is detachably connected to the light source device 350 and the processing device 330 by the connector 310d. Furthermore, as described later with reference to FIG. 4, a light guide 315 is inserted through the universal cable 310c, and the scope section 310 emits illumination light emitted from the light source device 350 from the leading end of the insertion section 310b through the light guide 315.

For example, the insertion section 310b has a distal end section, a curving section capable of curving, and a flexible tube from the leading end to the base end of the insertion section 310b. The insertion section 310b is inserted into an object. The distal end section of the insertion section 310b is the distal end section of the scope section 310, which is a hard distal end rigid section. An objective optical system 311 and the image sensor 312 described later are provided in the distal end section, for example.

The curving section can be curved in a desired direction in accordance with an operation to a curving operation member provided in the operation section 310a. The curving operation member includes, for example, a left/right curving operation knob and an up/down curving operation knob. In addition to the curving operation member, the operation section 310a may also be provided with various operation buttons, such as a release button and an air and water supply button.

The processing device 330 is a video processor that performs prescribed image processing to received imaging signals, thereby generating a captured image. Video signals of the generated captured image are output from the processing device 330 to the display section 340, and the live captured image is displayed on the display section 340. The configuration of the processing device 330 is described later. The display section 340 is, for example, a liquid crystal display or an EL (Electro-Luminescence) display.

The light source device 350 is a light source device capable of emitting white light for a normal observation mode. As described later in a second embodiment section, the light source device 350 may be capable of selectively emitting white light for the normal observation mode and second illumination light for generating a prediction image.

FIG. 4 is a diagram illustrating the configuration of each section of the endoscope system 300. Note that in FIG. 4, a part of the configuration of the scope section 310 is omitted and simplified.

The light source device 350 includes a light source 352 that emits illumination light. The light source 352 may be a xenon light source, LED (light emitting diode), or a laser light source. The light source 352 may also be other light sources, and an emission method is not limited.

The insertion section 310b includes the objective optical system 311, the image sensor 312, an illumination lens 314, and the light guide 315. The light guide 315 guides illumination light from the light source 352 to the leading end of the insertion section 310b. The illumination lens 314 irradiates an object with the illumination light guided by the light guide 315. The objective optical system 311 forms, as an object image, an image of the illumination light reflected from the object. The objective optical system 311 may include, for example, a focus lens, and may be capable of changing a position where the object image is formed depending on a position of the focus lens. For example, the insertion section 310b may include an actuator (not shown) which drives the focus lens based on control from a control section 332. In this case, the control section 332 performs AF (Auto Focus) control.

The image sensor 312 receives light from an object via the objective optical system 311. The image sensor 312 may be a monochrome sensor or an element equipped with a color filter. The color filter may be a widely known Bayer filter, a complementary color filter, or any other filter. The complementary color filter is a filter including color filters for each color of cyan, magenta, and yellow.

The processing device 330 performs control of image processing and the entire system. The processing device 330 includes a preprocessing section 331, the control section 332, a storage section 333, the prediction processing section 334, the detection processing section 335, and the postprocessing section 336. For example, the preprocessing section 331 corresponds to the acquisition section 110 of the image processing system 100. The prediction processing section 334 corresponds to the processing section 120 of the image processing system 100. Note that the control section 332, the detection processing section 335, the postprocessing section 336, etc. may be included in the processing section 120.

The preprocessing section 331 performs A/D conversion to convert an analog signal sequentially output from the image sensor 312 to a digital signal, and various correction processing to image data after the A/D conversion. Note that the image sensor 312 may be provided with an A/D conversion circuit, such that the A/D conversion in the preprocessing section 331 is omitted. The correction processing herein includes, for example, color matrix correction processing, structure enhancement processing, noise reduction processing, AGC (automatic gain control) or the like. Further, the preprocessing section 331 may perform other correction processing such as white balance processing. The preprocessing section 331 outputs a processed image as an input image to the prediction processing section 334 and the detection processing section 335. The preprocessing section 331 also outputs the processed image as a display image to the postprocessing section 336.

The prediction processing section 334 performs processing of estimating a prediction image based on an input image. For example, the prediction processing section 334 operates following the information about the trained model stored in the storage section 333 to perform processing of generating a prediction image.

The detection processing section 335 performs processing of detecting a region of interest from a detection target image. The detection target image herein is, for example, a prediction image estimated by the prediction processing section 334. The detection processing section 335 also outputs an estimation probability representing certainty of the detected region of interest. For example, the detection processing section 335 operates following the information about the trained model stored in the storage section 333 to perform detection processing.

Note that there may be one type of a region of interest in the present embodiment. For example, a region of interest may be a polyp and the detection processing may be to identify a position and a size of the polyp in the detection target image. Further, the region of interest in the present embodiment may include a plurality of types. For example, known is a method of classifying a polyp according to its state into TYPE1, TYPE2A, TYPE2B, and TYPE3. The detection processing in the present embodiment may include not only processing of simply detecting a position and a size of a polyp, but also processing of classifying the polyp into any of the above-mentioned types. In this case, the detection processing section 335 outputs information representing certainty of the classification results.

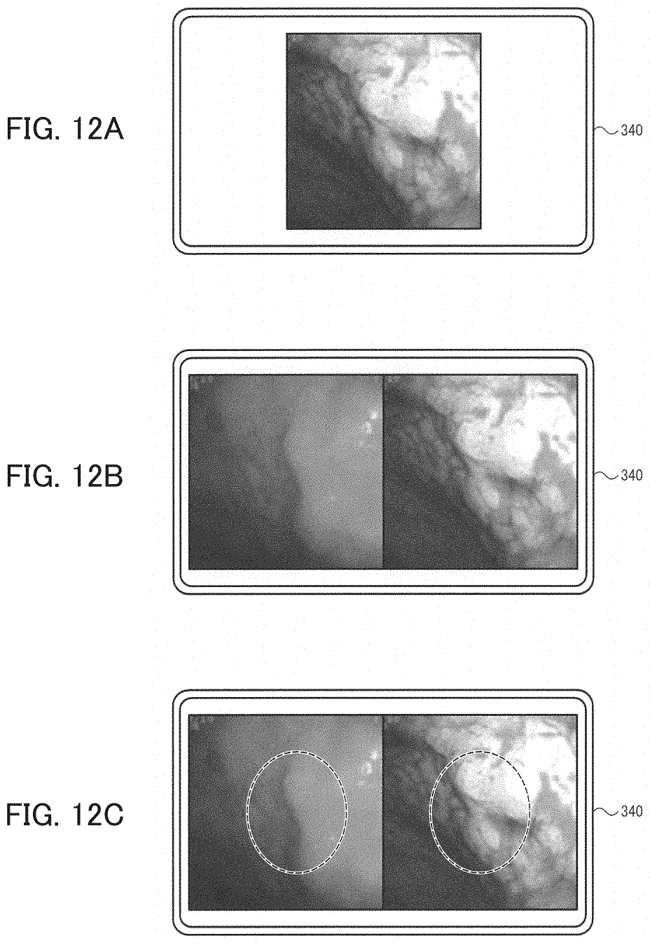

The postprocessing section 336 performs postprocessing based on outputs from the preprocessing section 331, the prediction processing section 334, and the detection processing section 335, and outputs a postprocessed image to the display section 340. For example, the postprocessing section 336 may acquire a white light image from the preprocessing section 331 and perform processing of displaying the white light image. The postprocessing section 336 may also acquire a prediction image from the prediction processing section 334 and perform processing of displaying the prediction image. Further, the postprocessing section 336 may perform processing of associating a display image with the prediction image and displaying the same. Further, the postprocessing section 336 may perform processing of adding detection results in the detection processing section 335 to the display image and the prediction image and displaying the images after the addition. An example display is described later with reference to FIGS. 12A to 12C.

The control section 332 is connected to the image sensor 312, the preprocessing section 331, the prediction processing section 334, the detection processing section 335, the postprocessing section 336, and the light source 352, and controls each section.

As described above, the image processing system 100 in the present embodiment includes the acquisition section 110 and the processing section 120. The acquisition section 110 obtains, as an input image, a biological image captured under the first imaging condition. The imaging condition herein is a condition under which an image of an object is to be captured, including various conditions that change imaging results, such as the illumination light, an imaging optical system, a position and orientation of the insertion section 310b, image processing parameters for a captured image, and processing to an object performed by a user. In a narrow sense, the imaging condition is a condition relating to illumination light or a condition relating to presence or absence of pigments dispersion. For example, the light source device 350 of the endoscope system 300 includes a white light source that emits white light, and the first imaging condition is a condition under which white light is used to capture an image of an object. The white light is a light including a wide range of wavelength components in visible light, for example, light including all of components of a red wavelength band, a green wavelength band, and a blue wavelength band. Further, the biological image herein is an image in which a living body is captured. The biological image may be an image in which inside of a living body is captured or an image in which tissues removed from a subject is captured.

The processing section 120 performs processing, based on association information about an association between a biological image captured under the first imaging condition and a biological image captured under the second imaging condition that differs from the first imaging condition, of outputting a prediction image corresponding to an image in which an object captured in an input image is to be captured under the second imaging condition.

The prediction image herein is an image estimated to be obtained if the object captured in the input image is captured under the second imaging condition. As a result, in some embodiments, it is not necessary to actually use the configuration for implementing the second imaging condition, and thus an image equivalent to the one captured under the second imaging condition can easily be obtained.

In that case, the above-mentioned association information is used in the method of the present embodiment. In other words, the association between images that means, if such an image is obtained under the first imaging condition, such an image is to be captured under the second imaging condition, is used. As such, if at least the association information is obtained in advance, the first imaging condition and the second imaging condition can flexibly be changed. For example, the second imaging condition may be a condition under which special light observation is performed, or a condition under which pigments dispersion is performed.

In the method in Japanese Unexamined Patent Application Publication No. 2012-70935, components corresponding to narrow band light are reduced on the assumption that white light and the narrow band light are simultaneously emitted. Hence, both of a light source for the narrow band light and a light source for the white light are essential. In the method in Japanese Unexamined Patent Application Publication No. 2016-2133, pigments dispersion is performed and a dedicated light source is required for obtaining an image in which dye is not visually recognized. In addition, the technique in Japanese Unexamined Patent Application Publication No. 2000-115553 performs processing based on an optical spectrum of an object. It does not consider an association between images and requires an optical spectrum of each object.

In a narrow sense, the association information in the present embodiment may be indicative of a trained model obtained through machine learning of a relationship between the first training image captured under the first imaging condition and the second training image captured under the second imaging condition. The processing section 120 performs processing of outputting a prediction image based on the trained model and the input image. By such application of machine learning, it is possible to improve estimation accuracy of the prediction image.

Furthermore, the method of the present embodiment can be applied to the endoscope system 300 including the image processing system 100. The endoscope system 300 includes an illumination section that irradiates an object with illumination light, an imaging section that outputs a biological image in which the object is captured, and an image processing section. The illumination section includes the light source 352 and an illumination optical system. The illumination optical system includes, for example, the light guide 315 and the illumination lens 314. The imaging section corresponds to the image sensor 312, for example. The image processing section corresponds to the processing device 330.

The image processing section of the endoscope system 300 obtains, as an input image, a biological image captured under the first imaging condition, and performs processing, based on the association information, of outputting a prediction image corresponding to an image in which an object captured in the input image is to be captured under the second imaging condition. In this way, the endoscope system 300 can be implemented, which can output, based on imaging under the first imaging condition, both an image associated with the first imaging condition and an image associated with the second imaging condition.

The light source 352 of the endoscope system 300 includes the white light source that emits white light. The first imaging condition in the first embodiment is an imaging condition for capturing an image of an object using the white light source. Since a white light image is a bright image having natural color tones, the endoscope system 300 that displays a white light image is widely used. As a result, in some embodiments, it is possible to obtain an image associated with the second imaging condition by using such widely used configuration. In this case, a configuration for emitting special light is not essential, nor a treatment such as pigments dispersion that increases a burden.

Note that the processing performed by the image processing system 100 in the present embodiment may be implemented as an image processing method. The image processing method obtains, as an input image, a biological image captured under the first imaging condition; obtains association information about an association between the biological image captured under the first imaging condition and the biological image captured under the second imaging condition that differs from the first imaging condition; and outputs, based on the input image and the association information, a prediction image corresponding to an image in which an object captured in the input image is to be captured under the second imaging condition.

Further, a biological image in the present embodiment is not limited to an image captured by the endoscope system 300. For example, a biological image may be an image of removed tissues captured by a microscope or the like. For example, the method of the present embodiment can be applied to a microscope system including the image processing system 100.

1.2 Example of Second Imaging Condition

A prediction image in the present embodiment may be an image in which given information included in an input image is enhanced. For example, the first imaging condition corresponds to a condition under which white light is used to capture an image of an object, and the input image corresponds to a white light image. The second imaging condition corresponds to an imaging condition under which given information can be enhanced as compared to the imaging condition using white light. In this way, it is possible to output, based on imaging with white light, an image with specific information being accurately enhanced.

More specifically, the first imaging condition corresponds to an imaging condition under which white light is used to capture an image of an object, and the second imaging condition corresponds to an imaging condition under which special light that differs in a wavelength band from the white light is used to capture an image of the object. Alternatively, the second imaging condition is an imaging condition under which pigments are to be dispersed to capture an image of the object. Hereinafter, for the convenience of description, the imaging condition under which white light is used to capture an image of an object is referred to as white light observation. The imaging condition under which special light is used to capture an image of an object is referred to as special light observation. The imaging condition under which pigments are to be dispersed to capture an image of an object is referred to as pigments dispersion observation. Further, an image captured by the white light observation is referred to as a white light image, an image captured by the special light observation is referred to as a special light image, and an image captured by the pigments dispersion observation is referred to as a pigments dispersed image.

The special light observation requires a light source for emitting special light. This makes the configuration of the light source device 350 complicated. In addition, to perform the pigments dispersion observation, it is necessary to disperse pigments on an object. When pigments are dispersed, it is not easy to immediately recover the state prior to the pigments dispersion, and the pigments dispersion itself increases a burden on a physician and a patient. As a result, in some embodiments, it is possible to assist a physician in performing diagnosis by displaying an image with certain information being enhanced, as well as simplifying the configuration of the endoscope system 300 and reducing a burden on a physician, etc.

Hereinafter, a specific method of the special light observation and the pigments dispersion observation will be described. However, a wavelength band used for the special light observation and pigments used for the pigments dispersion observation, etc. are not limited to those described below; various techniques are known. In other words, a prediction image output in the present embodiment is not limited to an image associated with the following imaging conditions, and can be extended to an image associated with an imaging condition using other wavelength bands or other agents, etc.

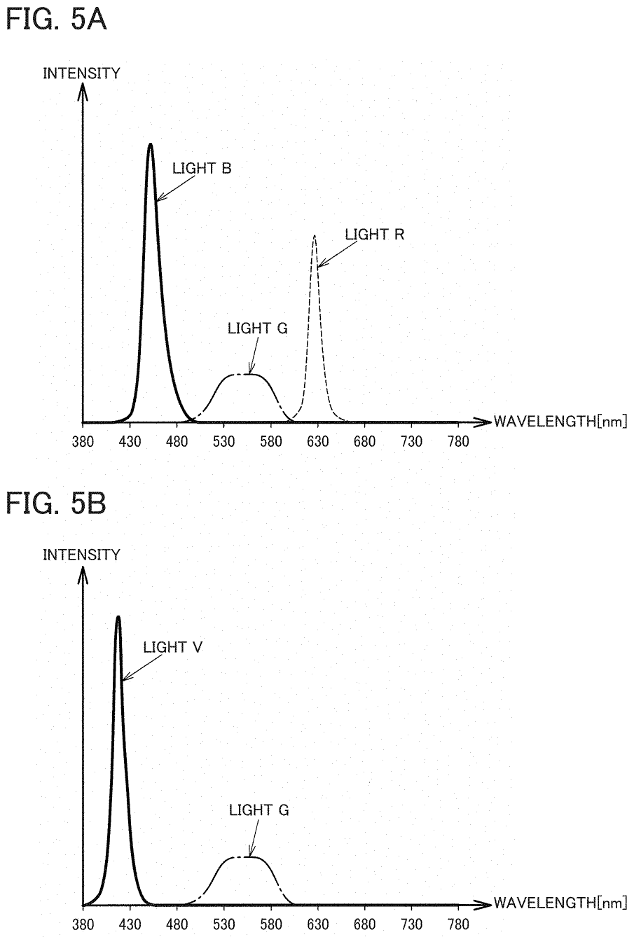

FIG. 5A illustrates an example of spectral characteristics of the light source 352 in the white light observation. FIG. 5B illustrates an example of spectral characteristics of irradiation light in NBI (Narrow Band Imaging), which is one example of the special light observation.

Light V is narrow band light with a peak wavelength of 410 nm. Half width of the light V is a few nm to tens of nm. The band of the light V belongs to a blue wavelength band of white light and is narrower than the blue wavelength band thereof. Light B is light having a blue wavelength band of white light. Light G is light having a green wavelength band of white light. Light R is light having a red wavelength band of white light. For example, the wavelength band of the light B is 430-500 nm, the wavelength band of the light G is 500-600 nm, and the wavelength band of the light R is 600-700 nm.

Note that the above wavelength is one example. For example, the peak wavelength of each light and the upper and lower bounds of the wavelength band may vary by about 10%. In addition, the light B, G, and R may be narrow band light with half width of a few nm to tens of nm.

At the time of the white light observation, as shown in FIG. 5A, the light B, G, and R are emitted but the light V is not. At the time of NBI, as shown in FIG. 5B, the light V and G are emitted, but the light B and R are not. The light V has a wavelength band absorbed by hemoglobin in blood. Using NBI enables observation of a vascular structure of a living body. In addition, by inputting an obtained signal to a certain channel, it is possible to display, in brown or the like, a lesion such as squamous cell carcinoma that is difficult to visually recognize under normal light, whereby preventing occurrence of a missed lesion site.

It is known that light of a wavelength band of 530 nm-550 nm is also easily absorbed by hemoglobin. Hence, in NBI, light G2 of a wavelength band of 530 nm-550 nm may be used. In this case, NBI is performed by emitting the light V and G2, but not the light B, G, or R.

As a result, in some embodiments, even if the light source device 350 does not include the light source 352 for emitting the light V or the light source 352 for emitting the light G2, it is possible to estimate a prediction image equivalent to those estimated by using NBI.

Further, the special light observation may be AFI. AFI is fluorescence imaging. In AFI, autofluorescence from fluorescent substances such as collagen can be observed by emitting excitation light, which is light of a wavelength band of 390 nm-470 nm. The autofluorescence corresponds to, for example, light of a wavelength band of 490 nm-625 nm. AFI can display a lesion enhanced with different color tones from that of the normal mucosa, whereby preventing occurrence of a missed lesion site.

Further, the special light observation may be IRI. IRI specifically uses a wavelength band of 790 nm-820 nm or 905 nm-970 nm. In IRI, ICG (indocyanine green) is intravenously injected and then irradiated with infrared light of the above wavelength band, ICG being an infrared indicator by which infrared light is easily absorbed. This enables enhanced information concerning blood vessels or blood flows in deep mucosa difficult to visually recognize by human eyes, allowing diagnosis of invasion depth and determination of a treatment policy, etc. of gastric cancer. Note that the numbers 790 nm-820 nm are obtained from the characteristics of the strongest absorption of the infrared indicator and the numbers 905 nm-970 nm are obtained from the characteristics of the weakest absorption of the infrared indicator. However, the wavelength band in this case is not limited thereto, and various modifications can be made to the upper and lower bounds of the wavelength, the peak wavelength, or the like.

Furthermore, the special light observation is not limited to NBI, AFI, or IRI. For example, the special light observation may be observation using the light V and A. The light V is suitable for obtaining characteristics of surface blood vessels of mucosa or a glandular structure. The light A is narrow band light with a peak wavelength of 600 nm, and half width thereof is a few nm to tens of nm. The band of the light A belongs to a red wavelength band of white light and is narrower than the red wavelength band thereof. The light A is suitable for obtaining characteristics of deep blood vessels or redness of mucosa, inflammation, etc. That is, the special light observation using the light V and A enables detection of presence of a wide variety of lesions such as cancer and inflammatory diseases.

Additionally, a contrast method, a staining method, a reaction method, a fluorescence method, intravascular dye injection, etc. are known as the pigments dispersion observation.

A contrast method is a method of enhancing surface unevenness of an object by utilizing a dye accumulation phenomenon. For example, dye such as indigo carmine is used for the contrast method.

A staining method is a method of observing a phenomenon that a dye solution stains biological tissues. For example, dye such as methylene blue and crystal violet is used for the staining method.

A reaction method is a method of observing a phenomenon that dye reacts specifically in a specific environment. For example, dye such as lugol is used for the reaction method.

A fluorescence method is a method of observing fluorescence expression of dye. For example, dye such as fluorestin is used for the fluorescence method.

Intravascular dye injection is a method of injecting dye into blood vessels to observe a phenomenon of coloring or staining of an organ or a vascular system due to the dye. For example, dye such as indocyanine green is used for the intravascular dye injection.

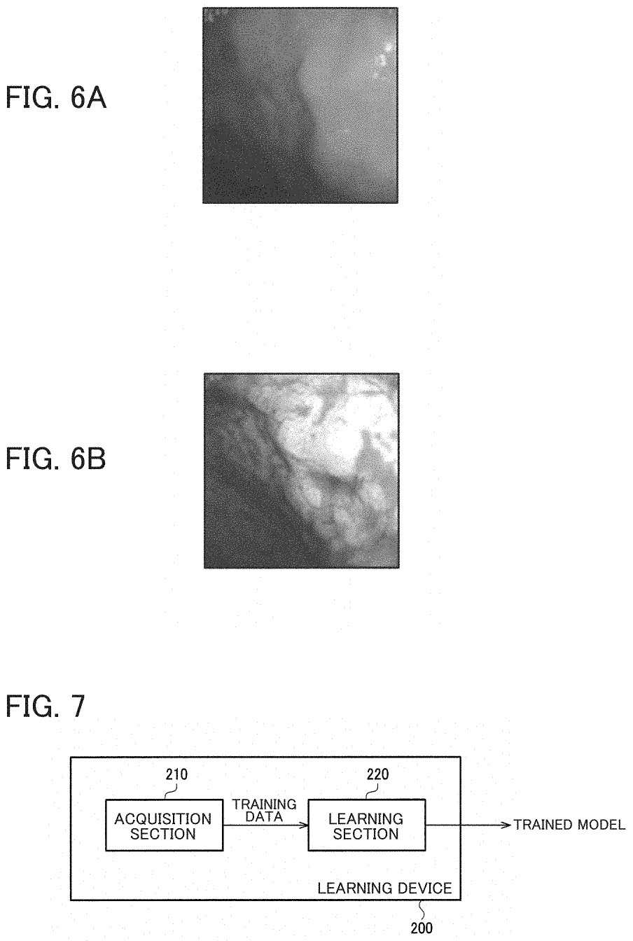

FIG. 6A illustrates an example of a white light image, and FIG. 6B illustrates an example of a pigments dispersed image obtained by using the contrast method. As shown in FIGS. 6A and 6B, the pigments dispersed image is an image with predetermined information being enhanced as compared to the white light image. Here, an example of the contrast method is illustrated, and thus the pigments dispersed image is an image with unevenness in the white light image being enhanced.

1.3 Learning Processing

FIG. 7 illustrates an example configuration of the learning device 200. The learning device 200 includes an acquisition section 210 and a learning section 220. The acquisition section 210 acquires the training data to be used for learning. One training data is data where input data is associated with a ground truth label corresponding to the input data. The learning section 220 generates a trained model through machine learning based on the acquired multiple training data. Details of the training data and a specific flow of the learning processing are described later.

The learning device 200 is an information processing device such as PC and a server system. Note that the learning device 200 may be implemented by distributed processing by a plurality of devices. For example, the learning device 200 may be implemented by cloud computing using a plurality of servers. Further, the learning device 200 may be configured integrally with the image processing system 100, or may be a separate device.

A summary of machine learning is to be described. Although machine learning using a neural network is described below, the method of the present embodiment is not limited thereto. In the present embodiment, for example, machine learning using other models such as a support vector machine (SVM) may be performed, or machine learning using a technique developed from various techniques such as a neural network and SVM may be performed.

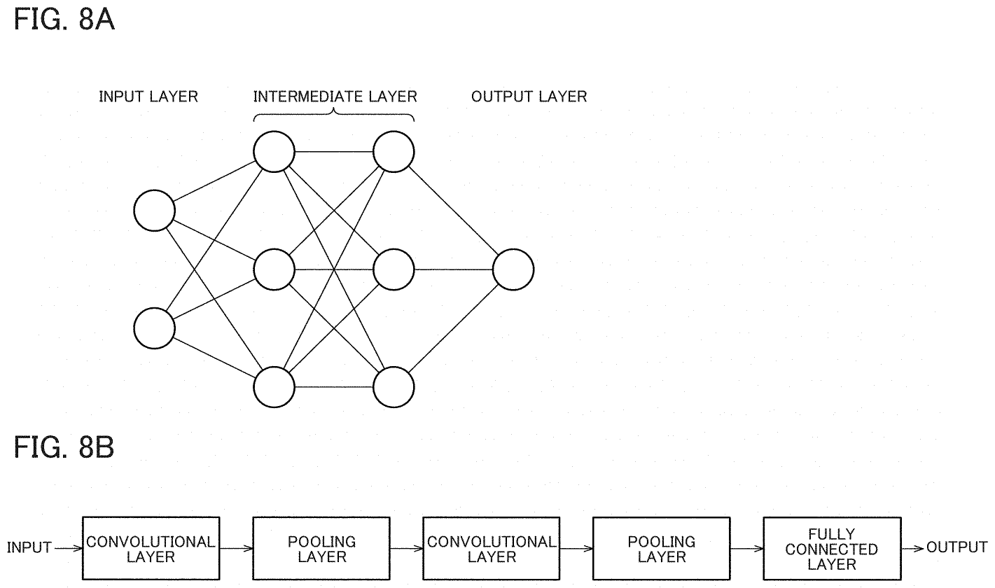

FIG. 8A is a schematic view describing a neural network. The neural network has an input layer to which data is input, an intermediate layer that performs an operation based on output from the input layer, and an output layer that outputs data based on output from the intermediate layer. While FIG. 8A illustrates a network with the two-layered intermediate layer, the intermediate layer may be one-layered, or 3-layered or more. In addition, the number of nodes included in each layer is not limited to the example in FIG. 8A, and can be implemented in various modifications. In consideration of accuracy, the learning in the present embodiment is preferably deep learning using a multilayer neural network. The multilayer herein means four or more layers, in a narrow sense.

As shown in FIG. 8A, a node included in a given layer is connected to a node in an adjacent layer. A weighting factor is set for each connection. Each node multiplies output of a previous node by the weighting factor to obtain the sum of the multiplication results. Further, each node adds bias to the sum and applies an activation function to the addition results, thereby obtaining the output of the node. By sequentially performing this processing from the input layer to the output layer, the output of the neural network is obtained. As the activation function, various functions such as a sigmoid function and an ReLU function are known, which are widely applicable to the present embodiment.

Learning in the neural network refers to processing of determining an appropriate weighting factor. The weighing factor herein includes bias. Specifically, the learning device 200 inputs the input data among the training data to the neural network, and performs a forward operation using the weighting factor at that time to obtain output. The learning section 220 of the learning device 200 calculates an error function based on the output and a ground truth label among the training data. Then, the weighting factor is updated to reduce the error function. For example, backpropagation can be used for updating the weighting factor, which updates the weighting factor from the output layer to the input layer.

Further, the neural network may be, for example, CNN (Convolutional Neural Network). FIG. 8B is a schematic view describing CNN. CNN includes a convolutional layer where a convolution operation is performed and a pooling layer. The convolutional layer is a layer where filter processing is performed. The pooling layer is a layer where a pooling operation is performed to reduce the size in vertical and horizontal directions. The example shown in FIG. 8B is a network that performs an operation in the convolutional layer and the pooling layer several times, and then performs an operation in a fully connected layer, thereby obtaining output. The fully connected layer is a layer where operation processing is performed if nodes in a given layer are connected to all nodes in a previous layer, the operation processing corresponding to the operation in each layer described above with reference to FIG. 8A. Although not shown in FIG. 8B, operation processing with an activation function is performed as in FIG. 8A also in the case of using CNN. Various configurations of CNN are known and can widely be applied to the present embodiment. Note that the output of the trained model in the present embodiment is, for example, a prediction image. Hence, CNN may include, for example, an inverse pooling layer. The inverse pooling layer is a layer where an inverse pooling operation is performed to increase the size in vertical and horizontal directions.

Also in the case of using CNN, the processing procedure is similar to those in FIG. 8A. That is, the learning device 200 inputs the input data among the training data to CNN, and performs filter processing using filter characteristics at that time and a pooling operation, thereby obtaining output. Based on the output and a ground truth label, an error function is calculated, and a weighting factor including the filter characteristics is updated to reduce the error function. For example, backpropagation can be used also for updating the weighting factor of CNN.

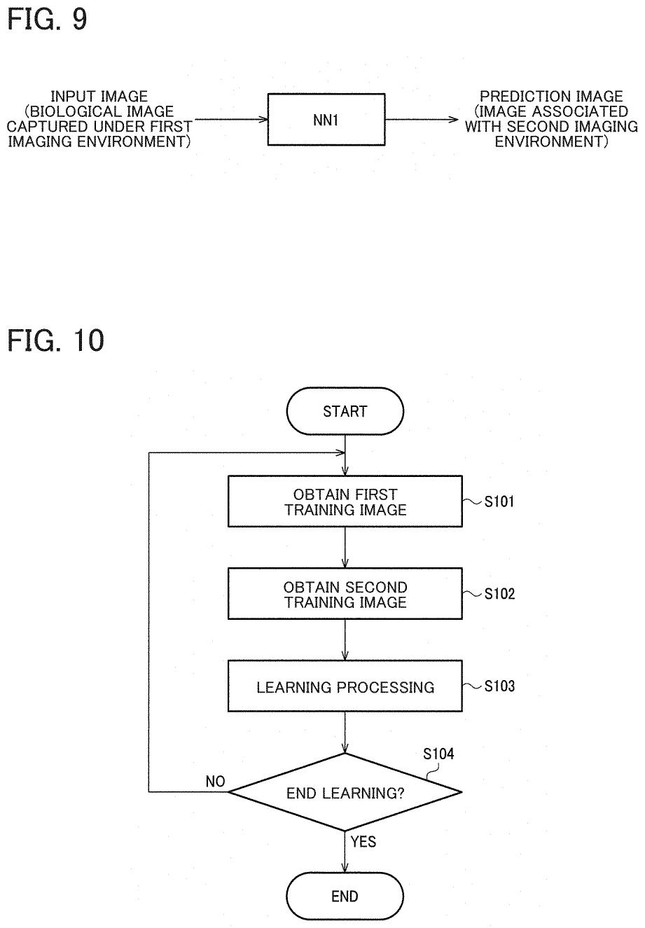

FIG. 9 is a diagram illustrating input and output of NN1, which is a neural network outputting a prediction image. As shown in FIG. 9, NN1 receives an input image as the input and performs a forward operation to output a prediction image. For example, the input image is a set of pixel values of xxyx3, wherein x is the number of vertical pixels, y is the number of horizontal pixels, and 3 is the number of channels of RGB. Similarly, the prediction image is also a set of pixel values of xxyx3. However, various modifications can be made to the number of pixels and the number of channels.

FIG. 10 is a flowchart describing learning processing of NN1. First, in steps 5101 and S102, the acquisition section 210 obtains the first training image and the second training image associated with the first training image. For example, the learning device 200 obtains, from the image gathering endoscope system 400, multiple data with the first training image being associated with the second training image, and stores the data as the training data in the storage section (not shown). The processing in the steps S101 and S102 are, for example, processing of reading out one of the training data.

The first training image is a biological image captured under the first imaging condition. The second training image is a biological image captured under the second imaging condition. For example, the image gathering endoscope system 400 is an endoscope system including a light source that emits white light and a light source that emits special light, and capable of obtaining both a white light image and a special light image. The learning device 200 obtains, from the image gathering endoscope system 400, data with the white light image being associated with the special light image in which the same object as the white light image is captured. Further, the second imaging condition may correspond to the pigments dispersion observation, and the second training image may be a pigments dispersed image.

In a step S103, the learning section 220 performs processing of obtaining an error function. Specifically, the learning section 220 inputs the first training image to NN1, and performs a forward operation based on a weighting factor at that time. Then, the learning section 220 obtains the error function based on comparison processing between the operation results and the second training image. For example, the learning section 220 obtains a difference absolute value of the pixel values for the operation results and each pixel of the second training image, and calculates an error function based on the sum or the mean, etc. of the difference absolute values. Further, in the step S103, the learning section 220 performs processing of updating the weighting factor to reduce the error function. This processing can utilize backpropagation or the like as described above. The processing in the steps S101-S103 correspond to a single learning processing based on one training data.

In a step S104, the learning section 220 determines whether or not to end the learning processing. For example, the learning section 220 may end the learning processing in case that the processing of the steps S101-S103 has been performed predetermined times. Alternatively, the learning device 200 may hold a part of the multiple training data as verification data. The verification data is data for confirming accuracy of learning results, and not used for updating the weighting factor. The learning section 220 may end the learning processing if an accuracy rate of estimation processing using the verification data is greater than a prescribed threshold value.

If No in the step S104, return to the step S101 and continue the learning processing based on next training data. If Yes in the step S104, end the learning processing. The learning device 200 transmits information about the generated trained model to the image processing system 100. In the example of FIG. 3, the information about the trained model is stored in the storage section 333. Note that various methods for machine learning such as batch learning and mini-batch learning are known and can be widely applied to the present embodiment.

The processing performed by the learning device 200 in the present embodiment may be implemented as a learning method. The learning method obtains the first training image, which is a biological image in which a given object is captured under the first imaging condition, and obtains the second training image, which is a biological image in which the given object is captured under the second imaging condition that differs from the first imaging condition. Then the learning method performs, based on the first training image and the second training image, machine learning of a condition for outputting a prediction image corresponding to an image in which the object included in the input image captured under the first imaging condition is to be captured under the second imaging condition.

1.4 Inference Processing

FIG. 11 is a flowchart illustrating processing of the image processing system 100 in the present embodiment. First, in a step S201, the acquisition section 110 obtains, as an input image, a biological image captured under the first imaging condition. For example, the acquisition section 110 obtains the input image that is a white light image.

In a step S202, the processing section 120 determines whether the current observation mode is the normal observation mode or an enhancement observation mode. The normal observation mode is an observation mode using a white light image. The enhancement observation mode is a mode in which given information included in a white light image is enhanced as compared to the normal observation mode. For example, the control section 332 of the endoscope system 300 determines the observation mode based on user input, and controls the prediction processing section 334, the postprocessing section 336, etc. according to the observation mode. As described later, however, the control section 332 may control to automatically change the observation mode based on various conditions.

If it is determined as the normal observation mode in the step S202, the processing section 120 performs processing, in a step S203, of displaying the white light image obtained in the step S201. For example, the postprocessing section 336 of the endoscope system 300 performs processing of displaying the white light image output from the preprocessing section 331 on the display section 340. Further, the prediction processing section 334 skips estimation processing of a prediction image.

On the other hand, if it is determined as the enhancement observation mode in the step S202, the processing section 120 performs processing, in a step S204, of estimating a prediction image. Specifically, the processing section 120 inputs the input image to the trained model NN1 to estimate the prediction image. Then, in a step S205, the processing section 120 performs processing of displaying the prediction image. For example, the prediction processing section 334 of the endoscope system 300 inputs the white light image output from the preprocessing section 331 to NNI to obtain the prediction image, NNI being the trained model read out from the storage section 333; and outputs the prediction image to the postprocessing section 336. The postprocessing section 336 performs processing of displaying an image including information about the prediction image, which is output from the prediction processing section 334, on the display section 340.

As shown in the steps S203 and S205 in FIG. 11, the processing section 120 performs processing of displaying at least one of the white light image captured using white light and the prediction image. Thus, by presenting the white light image having bright and natural color tones and the prediction image with different characteristics from the white light image, it is possible to present various information to a user. In that case, since no imaging under the second imaging condition is required, it is possible to simplify the system configuration and reduce a burden on a physician, etc.

FIGS. 12A to 12C illustrate examples of display screens of a prediction image. For example, the processing section 120 may perform processing of displaying a prediction image on the display section 340, as illustrated in FIG. 12A. FIG. 12A illustrates an example in which, for example, the second training image is a pigments dispersed image subjected to the contrast method, and the prediction image output from the trained model is an image corresponding to the pigments dispersed image. The same applies to FIGS. 12B and 12C.

Alternatively, the processing section 120 may perform processing, as illustrated in FIG. 12B, of displaying the white light image and the prediction image side by side. In this manner, the same object can be displayed in different ways, for example, so as to enable appropriate diagnosis support for a physician, etc. Since the prediction image is generated based on the white light image, there is no displacement of the object between images. Hence, it is easy for a user to associate the images with each other. Note that the processing section 120 may perform processing of displaying the entire white light image and the entire prediction image, or trimming at least one of the images.

Alternatively, the processing section 120 may display information concerning a region of interest included in the image, as shown in FIG. 12C. The region of interest in the present embodiment refers to a region that has a relatively high observation priority than the other regions for a user. If the user is a physician who performs diagnosis and treatment, the region of interest corresponds to, for example, a region where a lesion site is captured. However, if an object that the physician wants to observe is bubbles or residue, the region of interest may be a region where the bubbles or residue portion is captured. In other words, a target that should be noted by the user varies depending on the purpose of observation, and a region with a relatively high observation priority than other regions for the user during the observation is the region of interest.

In the example of FIG. 12C, the processing section 120 performs processing of displaying the white light image and the prediction image side by side, as well as displaying an elliptic object indicating the region of interest in each image. The detection processing of the region of interest may be performed, for example, using a trained model; the details of the processing is described later. The processing section 120 may also perform processing of superimposing a part of the prediction image corresponding to the region of interest in the white light image, and then perform processing of displaying the processing results. The displaying can be implemented in various modifications.

As described above, the processing section 120 of the image processing system 100 operates following a trained model to estimate a prediction image from an input image. The trained model herein corresponds to NN1.

The operation in the processing section 120 following a trained model, i.e. an operation for outputting output data based on input data, may be executed by software or hardware. In other words, a product-sum operation executed in each node in FIG. 8A, filter processing executed in a convolutional layer of CNN, etc. may be executed by software. Alternatively, the above operation may be executed by a circuit device such as FPGA. The above operation may also be executed by a combination of software and hardware. In this way, the action of the processing section 120 following the instructions from the trained model can be implemented in various ways. For example, the trained model includes inference algorithm and a weighting factor used in the inference algorithm. The inference algorithm is algorithm performing a filter operation or the like based on input data. In this case, both the inference algorithm and the weighting factor are stored in a storage section, and the processing section 120 may read out the inference algorithm and the weighting factor to perform the inference processing by software. The storage section is, for example, the storage section 333 of the processing device 330, but other storage sections may be used. Alternatively, the inference algorithm may be implemented by FPGA or the like, and the storage section may store the weighting factor. Alternatively, the inference algorithm including the weighting factor may be implemented by FPGA or the like. In this case, the storage section storing information about the trained model is, for example, a built-in memory of FPGA.

1.5 Selection of Trained Model

As described above, the second imaging condition may be the special light observation or the pigments dispersion observation. Furthermore, the special light observation includes a plurality of imaging conditions such as NBI. The pigments dispersion observation includes a plurality of imaging conditions such as the contrast method. The imaging condition associated with a prediction image in the present embodiment may be fixed to one given imaging condition. For example, the processing section 120 outputs a prediction image corresponding to an NBI image, but does not output a prediction image associated with other imaging conditions such as AFI. However, the method of the present embodiment is not limited thereto, and the imaging condition associated with the prediction image may be variable.

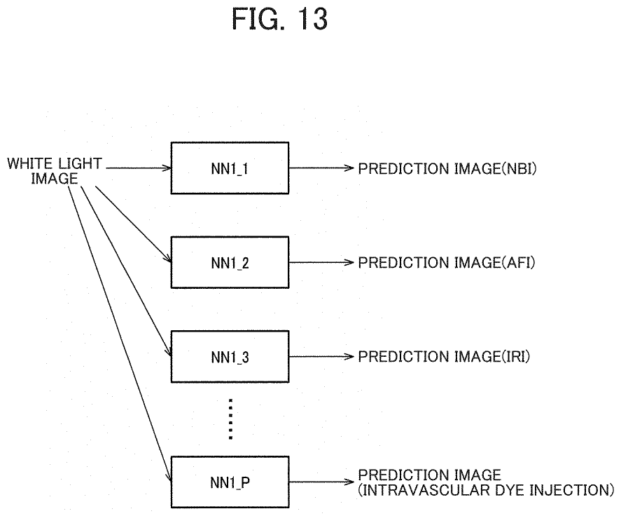

FIG. 13 is a diagram illustrating a specific example of the trained model NN1 that outputs a prediction image based on an input image. For example, NN1 may include a plurality of trained models NN1_1 to NN1_P that outputs the prediction images with different forms from each other. P is an integer of 2 or more.

The learning device 200 obtains, from the image gathering endoscope system 400, the training data with a white light image being associated with a special light image, the special light image being associated with NBI. Hereinafter, the special light image associated with NBI is referred to as an NBI image. Then, through machine learning based on the white light image and the NBI image, the trained model NN1_1 is generated that outputs, from the input image, a prediction image corresponding to the NBI image.

Likewise, NN1_2 is a trained model generated based on the training data with a white light image being associated with an AFI image, the AFI image being a special light image associated with AFI. NN1_3 is a trained model generated based on the training data with a white light image being associated with an IRI image, the IRI image being a special light image associated with IRI. NN1_P is a trained model generated based on the training data with a white light image being associated with a pigments dispersed image subjected to the intravascular dye injection.

The processing section 120 inputs the white light image as the input image to NN1_1, thereby obtaining the prediction image corresponding to the NBI image. The processing section 120 inputs the white light image as the input image to NN1_2, thereby obtaining a prediction image corresponding to the AFI image. The same applies to NN1_3 and the following ones; the processing section 120 changes the trained model to which the input image is to be input, to thereby change the prediction image.

For example, the image processing system 100 includes the normal observation mode and the enhancement observation mode as the observation mode, and includes a plurality of modes as the enhancement observation mode. The enhancement observation mode includes, for example, an NBI mode, an AFI mode, an IRI mode, and a mode associated with the light V and A, all of which are the special light observation mode. The enhancement observation mode also includes a contrast method mode, a staining method mode, a reaction method mode, a fluorescence method mode, and an intravascular dye injection mode, all of which are the pigments dispersion observation mode.

For example, a user selects any of the normal observation mode and the above plurality of enhancement observation modes. The processing section 120 operates in accordance with the selected observation mode. For example, if the NBI mode is selected, the processing section 120 reads out NN1_1 as the trained model, thereby outputting the prediction image corresponding to the NBI image.

Among the prediction images that can be output by the image processing system 100, a plurality of prediction images may simultaneously be output. For example, the processing section 120 may input a given input image to both NN1_1 and NN1_2, thereby performing processing of outputting the white light image, the prediction image corresponding to the NBI image, and the prediction image corresponding to the AFI image.

1.6 Diagnosis support

Described above is the processing of outputting a prediction image based on an input image. For example, a user, i.e. a physician, inspects a displayed white light image and a prediction image to perform diagnosis, etc. However, the image processing system 100 may present information concerning a region of interest to provide diagnosis support for a physician.

For example, as shown in FIG. 14A, the learning device 200 may detect a region of interest from a detection target image, and generate the trained model NN2 for outputting the detection results. The detection target image herein is a prediction image associated with a second imaging environment. For example, the learning device 200 obtains a special light image from the image gathering endoscope system 400 and obtains annotation results for the special light image. The annotation herein is processing of adding metadata to an image. The annotation results are information added by the annotation executed by a user. The annotation is performed by a physician, etc. who inspects an image as an annotation target. Note that the annotation may be performed in the learning device 200 or by other annotation devices.

When the trained model is a model performing processing of detecting a position of a region of interest, the annotation results include information that enables a position of the region of interest to be identified. For example, the annotation results include a detection frame and label information identifying an object included in the detection frame. When the trained model is a model performing processing of detecting types, the annotation results may be label information indicating the detection results of types. The detection results of types may be, for example, a result of classification into a lesion or a normal site, a result of classifying malignancy of a polyp in a prescribed stage, or other classification results. Hereinafter, the processing of detecting types is also referred to as classification processing. The detection processing in the present embodiment includes processing of detecting presence or absence of a region of interest, processing of detecting a position, the classification processing, etc.

The trained model NN2 that performs processing of detecting a region of interest may include a plurality of trained models NN2_1 to NN2_Q as shown in FIG. 14B. Q is an integer of 2 or more. The learning device 200 generates the trained model NN2_1 through machine learning based on the training data with an NBI image as the second training image being associated with the annotation results for the NBI image. Likewise, the learning device 200 generates NN2_2 based on an AFI image as the second training image and the annotation results for the AFI image. The same applies to NN2_3 and the following ones; the trained model for detecting a region of interest is provided for each type of an image as the input.

Provided herein is an example where one trained model is generated for one type of imaging condition, but not limited thereto. For example, a trained model for detecting a position of a region of interest from an NBI image and a trained model for the classification processing of a region of interest included in an NBI image may be generated separately. Further, the form of the detection results may differ depending on the image, such as, the trained model that performs processing of detecting the position of the region of interest is generated for an image associated with the light V and A, and the trained model that performs the classification processing is generated for the NBI image.

As described above, the processing section 120 may perform processing, based on a prediction image, of detecting a region of interest. Note that the processing section 120 is not precluded from detecting the region of interest based on a white light image. Furthermore, provided herein is an example where the trained model NN2 is used to perform the detection processing, but the method of the present embodiment is not limited thereto. For example, the processing section 120 may perform processing of detecting the region of interest based on feature amounts calculated from the image such as brightness, chroma, hue, and edge information. Alternatively, the processing section 120 may perform processing of detecting the region of interest based on image processing such as template matching.

In this manner, it is possible to present information about a region to be noted by a user, whereby enabling more appropriate diagnosis support. For example, the processing section 120 may perform processing of displaying an object representing a region of interest as illustrated in FIG. 12C.

The processing section 120 may also perform processing based on the results regarding a region of interest. Some specific examples are described below.

For example, the processing section 120 performs processing, based on a prediction image, of displaying information in case that a region of interest is detected. For example, as illustrated in FIG. 11, instead of performing division into the normal observation mode and the enhancement observation mode, the processing section 120 may always perform processing, based on a white light image, of estimating a prediction image. Then, the processing section 120 inputs the prediction image to NN2 to perform processing of detecting a region of interest. If the region of interest is not detected, the processing section 120 performs processing of displaying the white light image. That is, if there is no region such as a lesion, a bright and natural color image is preferentially displayed. On the other hand, if the region of interest is detected, the processing section 120 performs processing of displaying the prediction image. The prediction image can be displayed in various ways, as illustrated in FIGS. 12A to 12C. Since visibility of the region of interest is higher in the prediction image than the white light image, the region of interest such as a lesion is presented to a user in an easily visible manner

The processing section 120 may also perform processing based on certainty of the detection results. The trained models denoted by NN2_1 to NN2_Q can output the detection results indicating the position of the region of interest, as well as the information indicating the certainty of the detection results. Likewise, in case that the trained model outputs the classification results of the region of interest, the trained model can output the information indicating the certainty of the classification results. For example, if the output layer of the trained model is a publicly known softmax layer, the certainty corresponds to numerical data of 0-1 representing a probability.

For example, the processing section 120 outputs a plurality of different kinds of prediction images based on an input image and some or all of a plurality of trained models NN1_1 to NN1_P as illustrated in FIG. 13. Further, the processing section 120 obtains, based on a plurality of prediction images and some or all of a plurality of trained models NN2_1 to NN2_Q illustrated in FIG. 14B, the detection results of a region of interest and the certainty of the detection results for the respective prediction image. Then, the processing section 120 performs processing of displaying information concerning the prediction image with the most certain detection results of the region of interest. For example, if the detection results based on the prediction image corresponding to an NBI image is determined to be the most certain, the processing section 120 displays the prediction image corresponding to the NBI image and the detection results of the region of interest based on the prediction image. This enables the most suitable prediction image for diagnosis of the region of interest to become a display target. Furthermore, when displaying the detection results, the most reliable information can be displayed.

The processing section 120 may also perform processing according to a diagnosis scene as described below. For example, the image processing system 100 has a presence diagnosis mode and a qualitative diagnosis mode. As illustrated in FIG. 11, the observation mode is divided into the normal observation mode and the enhancement observation mode, and the enhancement observation mode may include the presence diagnosis mode and the qualitative diagnosis mode. Alternatively, as described above, estimation of a prediction image based on a white light image is always performed in the background, and processing on the prediction image may be divided into the presence diagnosis mode and the qualitative diagnosis mode.

In the presence diagnosis mode, the processing section 120 estimates, based on an input image, a prediction image associated with irradiation of the light V and A. As described above, this prediction image is an image suitable for detecting presence of a wide variety of lesions such as cancer and inflammatory diseases. The processing section 120 performs processing, based on the prediction image associated with irradiation of the light V and A, of detecting presence or absence and a position of a region of interest.

Further, in the qualitative diagnosis mode, the processing section 120 estimates, based on an input image, a prediction image corresponding to an NBI image or a pigments dispersed image. Hereinafter, the qualitative diagnosis mode in which the prediction image corresponding to the NBI image is output is referred to as an NBI mode, and the qualitative diagnosis mode in which the prediction image corresponding to the pigments dispersed image is output is referred to as a simulated staining mode.

The detection results in the qualitative diagnosis mode are, for example, qualitative support information concerning a lesion detected in the presence diagnosis mode. The qualitative support information can be supposed to be various information available for diagnosis of a lesion, such as, for example, progress of a lesion, or a degree of a symptom, a range of a lesion, or a boundary between a lesion and a normal area. For example, classification according to classification criteria established by academic societies or the like may be learned by a trained model, and the classification results by the trained model can be used as the support information.

The detection results in the NBI mode corresponds to the classification results classified according to various NBI classification criteria. The NBI classification criteria includes, for example, VS classification as classification criteria for gastric lesions, or JNET, NICE classification, and EC classification as classification criteria for colorectal lesions. Further, the detection results in the simulated staining mode corresponds to the detection results of a lesion according to classification criteria with staining. The learning device 200 generates a trained model through machine learning based on the annotation results according to these classification criteria.

FIG. 15 is a flowchart illustrating a processing procedure performed by the processing section 120 when switching from the presence diagnosis mode to the qualitative diagnosis mode. In a step S301, the processing section 120 sets the observation mode to the presence diagnosis mode. That is, the processing section 120 generates, based on an input image that is a white light image and NN1, a prediction image associated with irradiation of the light V and A. The processing section 120 also performs processing, based on the prediction image and NN2, of detecting a position of a region of interest.

Next, in a step S302, the processing section 120 determines whether or not a lesion indicated by the detection results has a predetermined area or greater. If the lesion has the predetermined area or greater, the processing section 120 sets the diagnosis mode to the NBI mode of the qualitative diagnosis mode in a step S303. If the lesion has an area smaller than the predetermined area, return to the step S301. That is, the processing section 120 displays a white light image if a region of interest is not detected. If the region of interest is detected but has an area smaller than a predetermined area, the information about the prediction image associated with irradiation of the light V and A is displayed. The processing section 120 may display only the prediction image, display the white light image and the prediction image side by side, or display the detection results based on the prediction image.

In the NBI mode in the step S303, the processing section 120 generates, based on the input image that is the white light image and NN1, a prediction image corresponding to an NBI image. The processing section 120 also performs processing of classifying the region of interest based on the prediction image and NN2.

Next, in a step S304, the processing section 120 determines, based on the classification results and the certainty of the classification results, whether or not further scrutiny is required. If the scrutiny is determined to be unnecessary, return to the step S302. If the scrutiny is determined to be required, the processing section 120 sets the simulated staining mode of the qualitative diagnosis mode in a step S305.

The step S304 will be described in detail. For example, in the NBI mode, the processing section 120 classifies the lesion detected in the presence diagnosis mode into Type1, Type2A, Type2B, and Type3. These types are classification characterized by a blood vessel pattern of mucosa and a surface structure of mucosa. The processing section 120 outputs a probability of the lesion being Type1, a probability of the lesion being Type2A, a probability of the lesion being Type2B, and a probability of the lesion being Type3.

The processing section 120 determines, based on the classification results in the NBI mode, whether discrimination of the lesion is difficult or not. For example, the processing section 120 determines that the discrimination is difficult if the probabilities of the lesion being Type1 or Type2A are equivalent to each other. In this case, the processing section 120 sets the simulated staining mode for simulatively reproducing indigo carmine staining.

In the simulated staining mode in the step S305, the processing section 120 outputs, based on the input image and the trained model NN1, a prediction image corresponding to a pigments dispersed image in which indigo carmine is to be dispersed. Further, the processing section 120 classifies the lesion as a hyperplastic polyp or a low-grade intramucosal tumor based on the prediction image and the trained model NN2. Such classification is characterized by a pit pattern in the indigo carmine stained image. In contrast, if the probability of the lesion being Type1 is a threshold value or greater, the processing section 120 classifies the lesion as a hyperplastic polyp, and does not make the shift to the simulated staining mode. Further, if the probability of the lesion being Type2A is a threshold value or greater, the processing section 120 classifies the lesion as a low-grade intramucosal tumor, and the processing section 120 does not make the shift to the simulated staining mode.

If the probabilities of the lesion being Type2A or Type2B are equivalent to each other, the processing section 120 determines that the discrimination is difficult. In this case, in the simulated staining mode in the step S305, the processing section 120 sets the simulated staining mode for simulatively reproducing crystal violet staining. In this simulated staining mode, the processing section 120 outputs, based on the input image, a prediction image corresponding to a pigments dispersed image in which crystal violet is to be dispersed. Further, the processing section 120 classifies the lesion, based on the prediction image, as a low-grade intramucosal tumor, or a high-grade intramucosal tumor, or a mild submucosal invasive carcinoma. Such classification is characterized by a pit pattern in the crystal violet stained image. If the probability of the lesion being Type2B is a threshold value or greater, the lesion is classified as a deep submucosal invasive carcinoma, and no shift to the simulated staining mode is made.

If Type2B and Type3 are difficult to discriminate, in the simulated staining mode in the step S305, the processing section 120 sets the simulated staining mode for simulatively reproducing crystal violet staining The processing section 120 outputs, based on the input image, the prediction image corresponding to the pigments dispersed image in which crystal violet is to be dispersed. Further, the processing section 120 classifies the lesion, based on the prediction image, as a high-grade intramucosal tumor or a mild submucosal invasive carcinoma or a deep submucosal invasive carcinoma.

Next, in a step S306, the processing section 120 determines whether or not the lesion detected in the step S305 has a predetermined area or greater. The determination method is the same as the step S302. If the lesion has the predetermined area or greater, return to the step S305. If the lesion has an area smaller than the predetermined area, return to the step S301.

While the above described is an example where the diagnosis mode transition is based on the detection results of the region of interest, the method of the present embodiment is not limited thereto. For example, the processing section 120 may determine the diagnosis mode based on a user operation. For example, when the leading end of the insertion section 310b of the endoscope system 300 is close to an object, it is considered that a user wants to observe the desired object in detail. Therefore, the processing section 120 may select a presence confirmation mode if distance to the object is a given threshold value or greater, and if the distance to the object becomes less than the threshold value, the shift to the qualitative diagnosis mode may be made. The distance to the object may be measured using a distance sensor, or determined using luminance of an image or the like. Additionally, various modifications can be made to the mode transition based on a user operation, such as, shifting to the qualitative diagnosis mode if the leading end of the insertion section 310b is faced to the object. Further, the prediction image used in a presence determination mode is not limited to the above-mentioned prediction image associated with the light V and A, and can be implemented in various modifications. Further, a prediction image to be used in the qualitative diagnosis mode is not limited to the above-mentioned prediction image corresponding to the NBI image or the pigments dispersed image, and can be implemented in various modifications.

As described above, the processing section 120 may be capable of outputting, based on a plurality of trained models and the input image, a plurality of different kinds of prediction images. A plurality of trained models is, for example, the above NN1_1 to NN1_P. Note that a plurality of trained models may be NN3_1 to NN3_3 or the like described later in the second embodiment section. Then, the processing section 120 performs processing, based on a given condition, of selecting the prediction image to be output among a plurality of prediction images. The processing section 120 herein corresponds to the detection processing section 335 or the postprocessing section 336 in FIG. 4. For example, by determining in the detection processing section 335 which trained model is to be used, the prediction image to be output may be selected. Alternatively, the detection processing section 335 may output a plurality of prediction images to the postprocessing section 336, and which prediction image is to be output to the display section 340 or the like may be determined in the postprocessing section 336. In this manner, it is possible to flexibly change the prediction image to be output.

The given condition herein includes at least one of a first condition relating to detection results of a position or a size of a region of interest based on a prediction image, a second condition relating to detection results of a type of a region of interest based on a prediction image, a third condition relating to certainty of a prediction image, a fourth condition relating to a diagnosis scene determined based on a prediction image, and a fifth condition relating to a part of an object captured in an input image.

For example, the processing section 120 obtains detection results based on at least one of the trained models NN2_1 to NN2_Q. The detection results herein may be the results of detection processing of detecting a position and a size in a narrow sense, or may be the results of classification processing of detecting a type. For example, if a region of interest is detected in any one of a plurality of prediction images, it is considered that the region of interest is captured in the prediction image in an easily recognizable manner Hence, the processing section 120 performs processing of preferentially outputting the prediction image in which the region of interest is detected. The processing section 120 may also perform processing, based on the classification processing, of preferentially outputting the prediction image in which the region of interest with higher severity is detected. In this manner, it is possible to output the appropriate prediction image according to the detection results.

Alternatively, as illustrated in FIG. 15, the processing section 120 may determine the diagnosis scene based on a prediction image, and select the prediction image to be output based on the diagnosis scene. The diagnosis scene represents a situation of diagnosis using a biological image, and includes, for example, a scene in which the presence diagnosis is performed or a scene in which the qualitative diagnosis is performed, as described above. For example, the processing section 120 determines the diagnosis scene based on the detection results of a region of interest in a given prediction image. By outputting a prediction image according to the diagnosis scene in this manner, it is possible to support user's diagnosis as appropriate.

Alternatively, as described above, the processing section 120 may select a prediction image to be output based on the certainty of the prediction image. In this manner, highly reliable prediction image can be a display target.

Alternatively, the processing section 120 may select a prediction image depending on a part of an object. An expected region of interest is different depending on the part as a diagnosis target. The imaging condition suitable for diagnosis of a region of interest is also different depending on the region of interest. That is, by changing a prediction image to be output depending on the part, it is possible to display the prediction image suitable for diagnosis.

Furthermore, the use of the conditions described above is not limited to the use of any one of the conditions, and 2 or more conditions may be combined.

### 2. Second Embodiment

2.1 Method of Present Embodiment

The system configuration of the second embodiment is the same as those in FIGS. 1-4. However, the illumination section in the present embodiment emits first illumination light which is white light and second illumination light that differs in at least one of light distribution and a wavelength band from the first illumination light. For example, the illumination section has a first illumination section that emits the first illumination light and a second illumination section that emits the second illumination light, as described below. As described above, the illumination section includes the light source 352 and the illumination optical system. The illumination optical system includes the light guide 315 and the illumination lens 314. However, a common illumination section may be used to emit the first illumination light and the second illumination light in a time-division manner, and the illumination section is not limited to the configuration below.

A white light image captured using white light is used for display, for example. On the other hand, an image captured using the second illumination light is used for estimation of a prediction image. In the method of the present embodiment, light distribution or a wavelength band of the second illumination light is set such that an image captured using the second illumination light has higher similarity with an image captured in the second imaging environment, relative to a white light image. The image captured using the second illumination light is referred to as an intermediate image. A specific example of the second illumination light is described below.

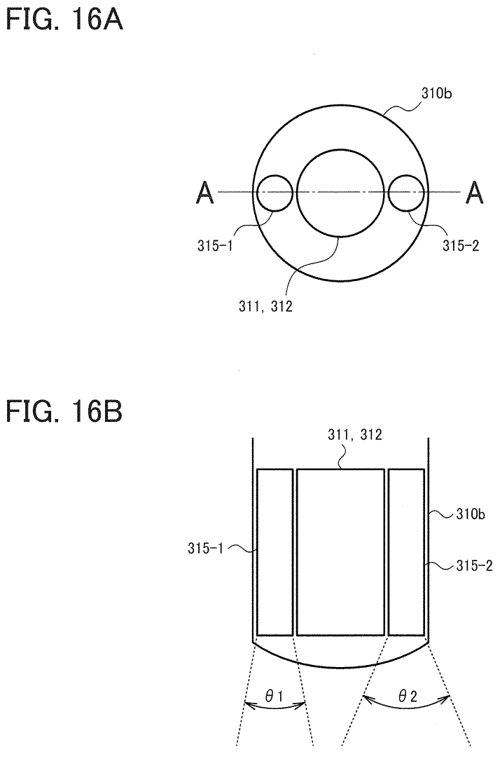

FIGS. 16A and 16B are diagrams illustrating the distal end section of the insertion section 310b when white light differs in light distribution from the second illumination light. The light distribution herein refers to information indicating a relationship between an irradiation direction and irradiation intensity of light. Wide light distribution means that a range irradiated with light having predetermined intensity or greater is wide. FIG. 16A is a diagram observing the distal end section of the insertion section 310b from a direction along the axis of the insertion section 310b. FIG. 16B is a sectional view along A-A in FIG. 16A.

As shown in FIGS. 16A and 16B, the insertion section 310b includes a first light guide 315-1 for emitting light from the light source device 350 and a second light guide 315-2 for emitting light from the light source device 350. In addition, though omitted in FIGS. 16A and 16B, the leading end of the first light guide 315-1 is provided with a first illumination lens as the illumination lens 314, and the leading end of the second light guide 315-2 is provided with a second illumination lens as the illumination lens 314.

Changing the shape of the leading end of the light guide 315 or the shape of the illumination lens 314 enables different light distribution. For example, the first illumination section includes the light source 352 that emits white light, the first light guide 315-1, and the first illumination lens. The second illumination section includes the given light source 352, the second light guide 315-2, and the second illumination lens. The first illumination section can irradiate an angle range of θ1 with illumination light having predetermined intensity or greater. The second illumination section can irradiate an angle range of θ2 with illumination light having predetermined intensity or greater. Here, θ1<θ2. That is, compared to the light distribution of the white light from the first illumination section, the light distribution of the second illumination light from the second illumination section is wider. Note that the light source 352 included in the second illumination section may be shared with the first illumination section, may be a part of a plurality of light sources included in the first illumination section, or may be other light sources not included in the first illumination section.

If using illumination light with narrow light distribution, a part of a biological image to be captured is bright and the rest is relatively dark. Since the observation of a biological image requires relatively high visibility of an entire image, a dynamic range covering a dark region to a bright region is set. Thus, when using the illumination light with narrow light distribution, 1LSB of pixel data corresponds to a certain brightness range. In other words, since value change of pixel data becomes smaller with respect to brightness change, surface unevenness on an object becomes less noticeable. In contrast, if using illumination light with wide light distribution, brightness of an entire image is relatively uniform. Thus, since value change of pixel data becomes greater with respect to brightness change, enhanced unevenness as compared to the narrow light distribution can be achieved.

As described above, by emitting the second illumination light with relatively wide light distribution, an unevenness enhanced image can be obtained, as compared to a white light image captured using a first irradiation section. Further, a pigments dispersed image subjected to the contrast method is an image in which unevenness of an object is enhanced. Accordingly, an image captured using illumination light with relatively wide light distribution is an image with higher similarity with a pigments dispersed image subjected to the contrast method, relative to a white light image. Therefore, by using an image captured using the illumination light with relatively wide light distribution as an intermediate image to estimate a prediction image based on the intermediate image, it is possible to improve the estimation accuracy compared to the case in which a prediction image is obtained directly from a white light image.

Furthermore, the white light emitted by the first illumination section and the second illumination light emitted by the second illumination section may differ in a wavelength band from each other. In this case, a first light source included in the first illumination section is different from a second light source included in the second illumination section. Alternatively, the first illumination section and the second illumination section may include filters that transmit a different wavelength band from each other, and share a common light source 352. Further, the light guide 315 and the illumination lens 314 may be provided separately in each of the first illumination section and the second illumination section, or may be shared by them.

For example, the second illumination light may be the light V. The light V has a relatively short wavelength band in the range of visible light, and does not reach the depths of a living body. Accordingly, an image obtained by irradiation by the light V includes a lot of information concerning a surface layer of a living body. In the pigments dispersion observation using the staining method, tissues in a surface layer of a living body are mainly stained. That is, an image captured using the light V has higher similarity with a pigments dispersed image subjected to the staining method relative to a white light image, and thus is usable as an intermediate image.

Alternatively, the second illumination light may be light of a wavelength band easily absorbed or reflected by a specific substance. The substance herein is, for example, glycogen. An image captured using a wavelength band easily absorbed or reflected by glycogen includes a lot of information about glycogen. Further, lugol is dye which reacts with glycogen, and in the pigments dispersion observation using the reaction method with lugol, glycogen is mainly enhanced. That is, an image captured using a wavelength band easily absorbed or reflected by glycogen has higher similarity with a pigments dispersed image subjected to the reaction method relative to a white light image, and thus is usable as an intermediate image.

Alternatively, the second illumination light may be illumination light associated with AFI. For example, the second illumination light is excitation light of a wavelength band of 390 nm-470 nm. AFI enhances an object similar to the one in a pigments dispersed image subjected to the fluorescence method with fluorestin. That is, an image captured using the illumination light associated with AFI has higher similarity with a pigments dispersed image subjected to the fluorescence method relative to a white light image, and thus is usable as an intermediate image.

As described above, the processing section 120 of the image processing system 100 according to the present embodiment performs processing of outputting, as a display image, a white light image captured under a display imaging condition under which white light is used to capture an image of an object. The first imaging condition in the present embodiment corresponds to an imaging condition that differs in at least one of light distribution and a wavelength band of the illumination light from the display imaging condition. In addition, the second imaging condition corresponds to an imaging condition under which special light that differs in a wavelength band from the white light is used to capture an image of an object, or an imaging condition under which pigments are to be dispersed to capture an image of an object.

The method of the present embodiment captures an intermediate image using the second illumination light that differs in light distribution or a wavelength band from the display imaging condition, and estimates a prediction image corresponding to a special light image or a pigments dispersed image based on the intermediate image.

For example, if the second imaging condition corresponds to the pigments dispersion observation as described above, it is possible to accurately obtain an image corresponding to a pigments dispersed image even in a situation where pigments are actually not dispersed. Compared to the case of irradiation only by white light, it is required to add the light guide 315, the illumination lens 314, the light source 352 or the like, but not required to consider dispersion or removal of an agent; thus, a burden on a physician or a patient can be reduced. Further, in the case of irradiation by the light V, the NBI observation is possible as shown in FIG. 5B. Hence, the endoscope system 300 may obtain a special light image by actually emitting special light, and obtain an image corresponding to a pigments dispersed image without performing pigments dispersion.

Furthermore, the prediction image estimated based on the intermediate image is not limited to an image corresponding to a pigments dispersed image. The processing section 120 estimates, based on the intermediate image, a prediction image corresponding to a special light image.

2.2 Learning Processing

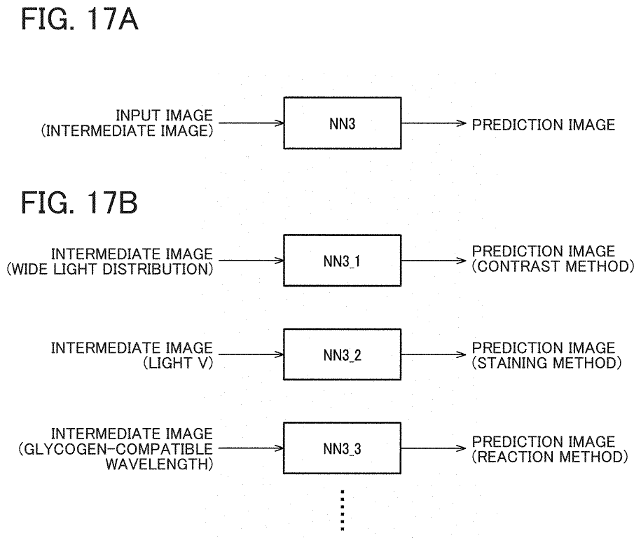

FIGS. 17A and 17B are diagrams illustrating input and output of a trained model NN3 to output a prediction image. As shown in FIG. 17A, the learning device 200 may generate, based on an input image, the trained model NN3 for outputting a prediction image. The input image in the present embodiment is an intermediate image captured using the second illumination light.

For example, the learning device 200 obtains, from the image gathering endoscope system 400 which can emit the second illumination light, the training data with the first training image in which a given object is captured using the second illumination light being associated with the second training image that is a special light image or a pigments dispersed image with the object captured therein. Based on the training data, the learning device 200 performs processing following the procedures described above with reference to FIG. 10, thereby generating the trained model NN3.

Further, FIG. 17B is a diagram illustrating a specific example of the trained model NN3 that outputs a prediction image based on an input image. For example, NN3 may include a plurality of trained models that outputs prediction images with different forms from each other. FIG. 17B illustrates NN3_1 to NN3_3 of a plurality of trained models.

The learning device 200 obtains, from the image gathering endoscope system 400, the training data where an image captured using the second illumination light with relatively wide light distribution is associated with a pigments dispersed image subjected to the contrast method. The learning device 200 generates, through machine learning based on the training data, the trained model NN3_1 that outputs, from an intermediate image, a prediction image corresponding to the pigments dispersed image subjected to the contrast method.

Likewise, the learning device 200 obtains the training data where an image captured using the second illumination light that is the light V is associated with a pigments dispersed image subjected to the staining method. The learning device 200 generates, through machine learning based on the training data, the trained model NN3_2 that outputs, from an intermediate image, a prediction image corresponding to the pigments dispersed image subjected to the staining method.

Likewise, the learning device 200 obtains the training data where an image captured using the second illumination light of a wavelength band easily absorbed or reflected by glycogen is associated with a pigments dispersed image subjected to the reaction method using lugol. The learning device 200 generates, through machine learning based on the training data, the trained model NN3_3 that outputs, from an intermediate image, a prediction image corresponding to the pigments dispersed image subjected to the reaction method.

As described above, the trained model NN3 that outputs a prediction image based on an intermediate image is not limited to NN3_1 to NN3_3, and can be implemented in other modifications.

2.3 Inference Processing

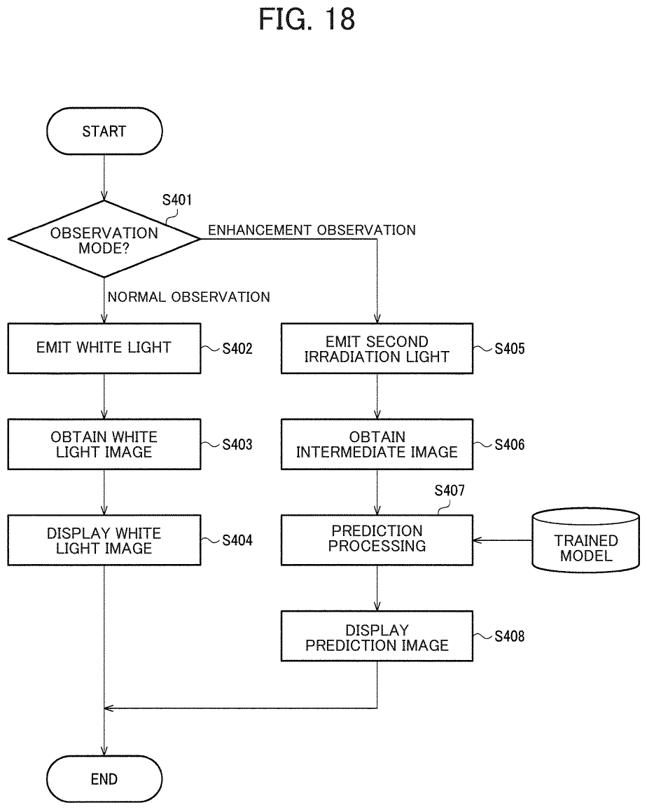

FIG. 18 is a flowchart illustrating processing of the image processing system 100 in the present embodiment. First, in a step S401, the processing section 120 determines whether the current observation mode is the normal observation mode or the enhancement observation mode. Similar to the example in FIG. 11, the normal observation mode is an observation mode using a white light image. The enhancement observation mode is a mode with given information included in a white light image being enhanced relative to the normal observation mode.

If determined as the normal observation mode in the step S401, the processing section 120 controls to emit white light in a step S402. Specifically, the processing section 120 herein corresponds to the control section 332 that executes control to capture an image under the display imaging condition using the first illumination section.

In a step S403, the acquisition section 110 obtains, as a display image, a biological image captured under the display imaging condition. For example, the acquisition section 110 obtains a white light image as the display image. In a step S404, the processing section 120 performs processing of displaying the white light image obtained in the step S402. For example, the postprocessing section 336 of the endoscope system 300 performs processing of displaying the white light image output from the preprocessing section 331 on the display section 340.

On the other hand, if determined as the enhancement observation mode in the step S401, the processing section 120 controls to emit the second illumination light in a step S405. Specifically, the processing section 120 herein corresponds to the control section 332 that executes control to capture an image under the first imaging condition using the second illumination section.

In a step S406, the acquisition section 110 obtains, as an input image, an intermediate image that is a biological image captured under the first imaging condition. In a step S407, the processing section 120 performs processing of estimating a prediction image. Specifically, the processing section 120 estimates the prediction image by inputting the input image to NN3. Then, in a step S408, the processing section 120 performs processing of displaying the prediction image. For example, the prediction processing section 334 of the endoscope system 300 inputs the intermediate image output from the preprocessing section 331 to NN3 to obtain the prediction image, NN3 being a trained model read out from the storage section 333; and outputs the prediction image to the postprocessing section 336. The postprocessing section 336 performs processing of displaying an image including information about the prediction image output from the prediction processing section 334 on the display section 340. As shown in FIGS. 12A to 12C, the displaying can be implemented in various modifications.

Similar to the first embodiment, the normal observation mode and the enhancement observation mode may be switched based on a user operation. Alternatively, the normal observation mode and the enhancement observation mode may be alternately executed.

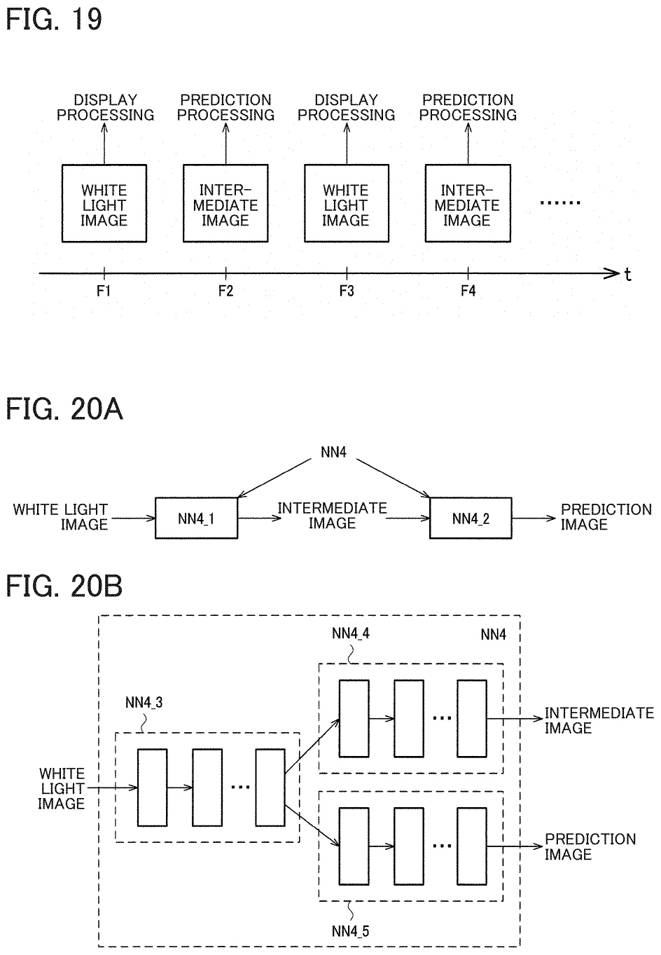

FIG. 19 is a diagram illustrating irradiation timing of white light and the second illumination light. The horizontal axis in FIG. 19 represents time and F1 to F4 respectively correspond to imaging frames of the image sensor 312. The white light is emitted in F1 and F3, and the acquisition section 110 obtains a white light image. In F2 and F4, the second illumination light is emitted and the acquisition section 110 obtains an intermediate image. The same applies to the subsequent frames; the white light and the second illumination light are alternately emitted.

As shown in FIG. 19, the illumination section irradiates an object with the first illumination light in the first imaging frame, and irradiates the object with the second illumination light in a second imaging frame that differs from the first imaging frame. In this way, the intermediate image can be obtained in an imaging frame different from the imaging frame of the white light image. However, the imaging frame irradiated with the white light and the imaging frame irradiated with the second illumination light may not be overlapped with each other; the specific order and frequency are not limited to those in FIG. 19 and can be implemented in various modifications.

Then, the processing section 120 performs processing of displaying the white light image that is a biological image captured in the first imaging frame. The processing section 120 also performs processing, based on an input image captured in the second imaging frame and association information, of outputting a prediction image. The association information is indicative of a trained model as described above. For example, when performing processing illustrated in FIG. 19, the white light image and the prediction image will be obtained once every 2 frames, respectively.

For example, similar to the example described above in the first embodiment section, the processing section 120 may display the white light image while performing processing of detecting a region of interest using the prediction image in the background. The processing section 120 performs processing of displaying the white light image until the region of interest is detected, and once the region of interest is detected, displays information based on the prediction image.

Note that the second illumination section may emit a plurality of illumination lights with different light distribution or a wavelength band from each other. The processing section 120 may be capable of outputting a plurality of different kinds of prediction images by switching the illumination light to be emitted among a plurality of illumination lights. For example, the endoscope system 300 may be capable of emitting white light, illumination light with wide light distribution, and the light V. In this case, the processing section 120 can output, as a prediction image, an image corresponding to a pigments dispersed image subjected to the contrast method, and an image corresponding to a pigments dispersed image subjected to the staining method. This enables accurate estimation of various prediction images.

As shown in FIG. 17B, in the present embodiment, the second illumination light is associated with the type of the prediction image predicted based on the second illumination light. Accordingly, the processing section 120 executes control based on an association between the illumination light and the trained model NN3 to be used for prediction processing. For example, if the processing section 120 controls to emit the illumination light with wide light distribution, it uses the trained model NN3_1 to estimate the prediction image, and if it controls to emit the light V, it uses the trained model NN3_2 to estimate the prediction image.

Also in the present embodiment, the processing section 120 may be capable of outputting a plurality of different kinds of prediction images based on a plurality of trained models and an input image. A plurality of trained models is, for example, NN3_1 to NN3_3. The processing section 120 performs processing, based on a given condition, of selecting the prediction image to be output among a plurality of prediction images. The given condition herein is, for example, the first to fifth conditions described above in the first embodiment.

In the present embodiment, the first imaging condition includes a plurality of imaging conditions under which different illumination light with different light distribution or a wavelength band is used for imaging, and the processing section 120 is capable of outputting, based on a plurality of trained models and the input image captured using the different illumination light, a plurality of different kinds of prediction images. The processing section 120 controls to change the illumination light based on a given condition. More specifically, the processing section 120 determines, based on the given condition, which illumination light among a plurality of illumination lights that can be emitted by the second illumination section is to be emitted to a second irradiation section. In this manner, also in the second embodiment in which the second illumination light is used to generate the prediction images, it is possible to change the prediction image to be output depending on a situation.

### 3. Third Embodiment

In the second embodiment, described is an example in which the image processing system 100 can obtain a white light image and an intermediate image. However, the intermediate image may be used in a learning phase. In the present embodiment, a prediction image is estimated based on the white light image, similar to the first embodiment.

The association information in the present embodiment may be indicative of a trained model obtained through machine learning of a relationship between the first training image captured under the first imaging condition, the second training image captured under the second imaging condition, and a third training image captured under a third imaging condition which differs from both the first imaging condition and the second imaging condition. The processing section 120 outputs a prediction image based on the trained model and an input image.

The first imaging condition corresponds to an imaging condition under which white light is used to capture an image of an object. The second imaging condition corresponds to an imaging condition under which special light that differs in a wavelength band from the white light is used to capture an image of the object, or an imaging condition under which pigments are to be dispersed to capture an image of the object. The third imaging condition corresponds to an imaging condition that differs in at least one of light distribution and a wavelength band of illumination light from the first imaging condition. In this way, it is possible to estimate a prediction image based on a relationship between a white light image, the prediction image, and an intermediate image.

FIGS. 20A and 20B illustrate examples of a trained model NN4 in the present embodiment. NN4 is a trained model that receives a white light image as input and outputs a prediction image based on the relationship between three images, i.e. the white light image, the intermediate image, and the prediction image.

As illustrated in FIG. 20A, NN4 may include a first trained model NN4_1 obtained through machine learning of a relationship between the first training image and the third training image, and a second trained model NN4_2 obtained through machine learning of a relationship between the third training image and the second training image.

For example, the image gathering endoscope system 400 is a system capable of emitting white light, the second illumination light, and special light, and capable of obtaining a white light image, an intermediate image, and a special light image. Further, the image gathering endoscope system 400 may be capable of obtaining a pigments dispersed image. The learning device 200 generates NN4_1 through machine learning based on the white light image and the intermediate image. The learning section 220 inputs the first training image to NN4_1 and performs a forward operation based on a weighting factor at that time. The learning section 220 obtains an error function based on comparison processing between the operation results and the third training image. The learning section 220 performs processing of updating the weighting factor to reduce the error function, thereby generating the trained model NN4_1.

Likewise, the learning device 200 generates NN4_2 through machine learning based on the intermediate image and the special light image, or the intermediate image and the pigments dispersed image. The learning section 220 inputs the third training image to NN4_2, and performs a forward operation based on a weighting factor at that time. The learning section 220 obtains an error function based on comparison processing between the operation results and the second training image. The learning section 220 performs processing of updating the weighting factor to reduce the error function, thereby generating the trained model NN4_2.

The acquisition section 110 obtains, as an input image, a white light image similar to the first embodiment. The processing section 120 generates, based on the input image and the first trained model NN4_1, an intermediate image corresponding to an image in which an object captured in the input image is to be captured under the third imaging condition. The intermediate image is an image corresponding to the intermediate image in the second embodiment. Then, the processing section 120 outputs a prediction image based on the intermediate image and the second trained model NN4_2.

As described above in the second embodiment section, the intermediate image captured by using the second illumination light is an image more similar to a special light image or a pigments dispersed image, than a white light image. Hence, compared to machine learning of only a relationship between a white light image and a special light image, or only a relationship between a white light image and a pigments dispersed image, it is possible to improve the estimation accuracy of a prediction image. If using the configuration illustrated in FIG. 20A, the input in the estimation processing of a prediction image is a white light image, and there is no need to emit the second illumination light in an estimation processing phase. Hence, it is possible to simplify the configuration of the illumination section.

Further, the configuration of the trained model NN4 is not limited to the one in FIG. 20A. For example, as shown in FIG. 20B, the trained model NN4 may include a feature amount extraction layer NN4_3, an intermediate image output layer NN4_4, and a prediction image output layer NN4_5. Note that the rectangles in FIG. 20B respectively represent one layer in the neural network. The layer herein is, for example, a convolutional layer or a pooling layer. The learning section 220 inputs the first training image to NN1 and performs a forward operation based on a weighting factor at that time. The learning section 220 obtains an error function based on comparison processing between the output of the intermediate image output layer NN4_4 among the operation results and the third training image, and comparison processing between the output of the prediction image output layer NN4_5 among the operation results and the second training image. The learning section 220 performs processing of updating the weighting factor to reduce the error function, thereby generating the trained model NN4.

Also in the case of using the configuration in FIG. 20B, machine learning in consideration of the relationship between three images is performed, and thus estimation accuracy of a prediction image can be improved. Further, the input of the configuration illustrated in FIG. 20B is a white light image, and there is no need to emit the second illumination light in the estimation processing phase. Thus, it is possible to simplify the configuration of the illumination section. In addition, various modifications can be made to the configuration of the trained model NN4 at the time of machine learning of the relationship between the white light image, the intermediate image, and the prediction image.

### 4. Modifications

Some modifications will be described below.

4.1 First Modification

In the third embodiment, described is an example of the endoscope system 300 having a configuration similar to the one in the first embodiment, estimating a prediction image based on a white light image. However, combination of the second embodiment and the third embodiment is also possible.

The endoscope system 300 can emit white light and the second illumination light. The acquisition section 110 of the image processing system 100 obtains a white light image and an intermediate image. The processing section 120 estimates a prediction image based on both the white light image and the intermediate image.

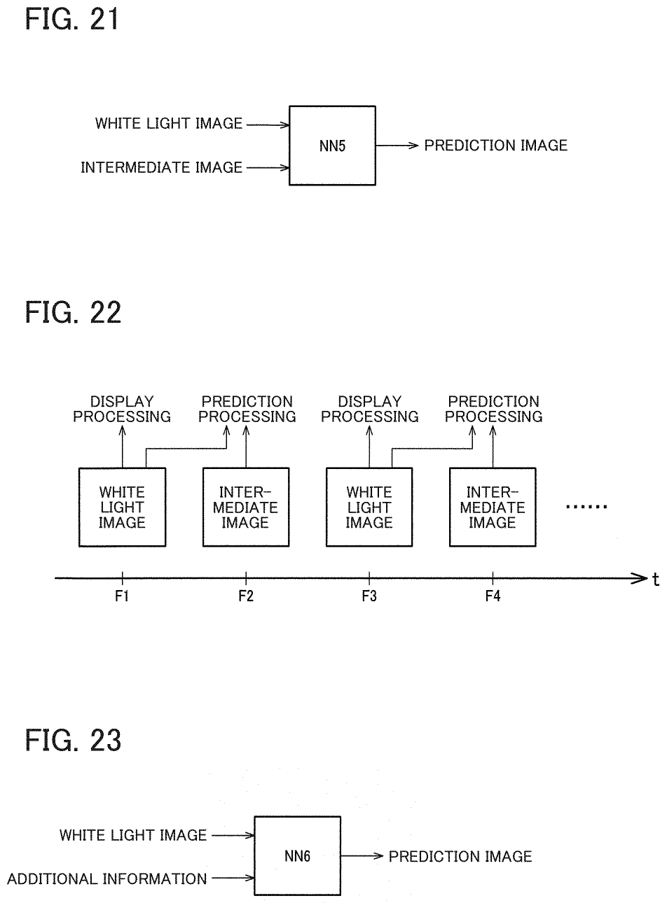

FIG. 21 is a diagram illustrating input and output of a trained model NN5 in the present modification. The trained model NN5 receives, as an input image, a white light image and an intermediate image, and outputs a prediction image based on the input image.

For example, the image gathering endoscope system 400 is a system capable of emitting white light, the second illumination light, and special light, and capable of obtaining a white light image, an intermediate image, and a special light image. Further, the image gathering endoscope system 400 may be capable of obtaining a pigments dispersed image. The learning device 200 generates NN5 through machine learning based on the white light image, the intermediate image, and the prediction image. Specifically, the learning section 220 inputs the first training image and the third training image to NN5 and performs a forward operation based on a weighting factor at that time. The learning section 220 obtains an error function based on comparison processing between the operation results and the second training image. The learning section 220 performs processing of updating the weighting factor to reduce the error function, thereby generating the trained model NN5.

The acquisition section 110 obtains a white light image and an intermediate image, similar to the second embodiment. The processing section 120 outputs a prediction image based on the white light image, the intermediate image, and the trained model NN5.

FIG. 22 is a diagram illustrating a relationship between imaging frames of the white light image and the intermediate image. Similar to the example in FIG. 19, the white light image is obtained in the imaging frames F1 and F3, and the intermediate image is obtained in F2 and F4. In the present modification, for example, a prediction image is estimated based on the white light image captured in F1 and the intermediate image captured in F2. Similarly, the prediction image is estimated based on the white light image captured in F3 and the intermediate image captured in F4. Also in this case, similar to the second embodiment, the white light image and the prediction image will be obtained once every 2 frames, respectively.

4.2 Second Modification

FIG. 23 is a diagram illustrating input and output of a trained model NN6 in another modification. The trained model NN6 is a model obtained through machine learning of a relationship between the first training image, additional information, and the second training image. The first training image is a white light image. The second training image is a special light image or a pigments dispersed image.

The additional information includes information concerning surface unevenness, information indicating an imaged part, information indicating a state of mucosa, information indicating a fluorescence spectrum of pigments to be dispersed, information concerning blood vessels, or the like.

Since the information concerning unevenness is indicative of an enhanced structure by the contrast method, using the information as the additional information can improve the estimation accuracy of a prediction image corresponding to a pigments dispersed image subjected to the contrast method.

In the staining method, presence or absence, distribution, a shape, etc. of a tissue to be stained are different depending on an imaged part, for example, depending on which part of an organ of a living body is captured. Hence, using the information indicating an imaged part as the additional information can improve the estimation accuracy of a prediction image corresponding to a pigments dispersed image subjected to the staining method.

In the reaction method, reaction of dye varies depending on a state of mucosa. Hence, using the information indicating a state of mucosa as the additional information can improve the estimation accuracy of a prediction image corresponding to a pigments dispersed image subjected to the reaction method.

Since fluorescence expression of dye is to be observed by the fluorescence method, observation of fluorescence on an image varies depending on a fluorescence spectrum. Hence, using the information indicating a fluorescence spectrum as the additional information can improve the estimation accuracy of a prediction image corresponding to a pigments dispersed image subjected to the fluorescence method.

In the intravascular dye injection and NBI, blood vessels are enhanced. Hence, using the information concerning blood vessels as the additional information can improve the estimation accuracy of a prediction image corresponding to a pigments dispersed image subjected to the intravascular dye injection or a prediction image corresponding to an NBI image.

The learning device 200 obtains, for example, control information at the time when the image gathering endoscope system 400 captured the first training image and the second training image, or the annotation results from a user, or the results of image processing to the first training image, as the additional information. The learning device 200 generates a trained model based on the training data where the first training image, the second training image, and the additional information are associated with each other. Specifically, the learning section 220 inputs the first training image and the additional information to the trained model, and performs a forward operation based on a weighting factor at that time. The learning section 220 obtains an error function based on comparison processing between the operation results and the second training image. The learning section 220 performs processing of updating the weighting factor to reduce the error function, thereby generating the trained model.

The processing section 120 of the image processing system 100 inputs an input image that is a white light image and the additional information to the trained model, thereby outputting a prediction image. The additional information may be obtained from control information about the endoscope system 300 at the time of capturing the input image, may be input by a user, or may be obtained by image processing to the input image.

4.3 Third Modification

Further, the association information is not limited to the trained model. In other words, the method of the present embodiment is not limited to those using machine learning.

For example, the association information may be a database including a plurality of pairs of a biological image captured under the first imaging condition and a biological image captured under the second imaging condition. For example, the database includes a plurality of pairs of a white light image and an NBI image in which the same object is captured. The processing section 120 compares an input image with the white light images included in the database to search the white light image with highest similarity with the input image. The processing section 120 outputs the NBI image associated with the searched white light image. In this way, it is possible to output a prediction image corresponding to the NBI image based on the input image.

The database may also be a database in which a white light image is associated with a plurality of images such as an NBI image, an AFI image, and an IRI image. In this way, the processing section 120 can output, based on the white light image, various prediction images such as a prediction image corresponding to the NBI image, a prediction image corresponding to the AFI image, and a prediction image corresponding to the IRI image. Which prediction image is to be output may be determined based on a user input, or based on the detection results of a region of interest, as described above.

Further, the image to be stored in the database may be an image obtained by subdividing one captured image. The processing section 120 divides an input image into a plurality of regions, and performs processing of searching an image with high similarity for each region from the database.

Furthermore, the database may be a database in which an intermediate image is associated with an NBI image or the like. In this way, the processing section 120 can output a prediction image based on an input image that is the intermediate image.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. An image processing system comprising a processor including hardware, the processor being configured to:
obtain, as an input image, a biological image captured under a first imaging condition; and
perform processing, based on association information of an association between the biological image captured under the first imaging condition and the biological image captured under a second imaging condition that differs from the first imaging condition, of outputting a prediction image corresponding to an image in which an object captured in the input image is to be captured under the second imaging condition, wherein
the association information is indicative of a trained model obtained through machine learning of a relationship between a first training image captured under the first imaging condition and a second training image captured under the second imaging condition,
the processor is capable of outputting, based on a plurality of the trained models and the input image, a plurality of different kinds of the prediction images, and
the processor performs processing, based on a given condition, of selecting the prediction image to be output among a plurality of the prediction images.

2. The image processing system as defined in claim 1, wherein
the first imaging condition corresponds to an imaging condition under which white light is used to capture an image of the object, and
the second imaging condition corresponds to an imaging condition under which special light that differs in a wavelength band from the white light is used to capture an image of the object, or to an imaging condition under which pigments are to be dispersed to capture an image of the object.

3. The image processing system as defined in claim 1, wherein
the processor performs processing of outputting, as a display image, a white light image captured under an display imaging condition under which white light is used to capture an image of the object,
the first imaging condition corresponds to an imaging condition that differs in at least one of light distribution and a wavelength band of illumination light from the display imaging condition, and
the second imaging condition corresponds to an imaging condition under which special light that differs in a wavelength band from the white light is used to capture an image of the object, or to an imaging condition under which pigments are to be dispersed to capture an image of the object.

4. The image processing system as defined in claim 1, wherein
the association information is indicative of a trained model obtained through machine learning of a relationship between the first training image captured under the first imaging condition, the second training image captured under the second imaging condition, and a third training image captured under a third imaging condition that differs from both the first imaging condition and the second imaging condition, and
the processor performs processing, based on the trained model and the input image, of outputting the prediction image.

5. The image processing system as defined in claim 4, wherein
the first imaging condition corresponds to an imaging condition under which white light is used to capture an image of the object,
the second imaging condition corresponds to an imaging condition under which special light that differs in a wavelength band from the white light is used to capture an image of the object, or to an imaging condition under which pigments are to be dispersed to capture an image of the object, and
the third imaging condition corresponds to an imaging condition that differs in at least one of light distribution and a wavelength band of illumination light from the first imaging condition.

6. The image processing system as defined in claim 4, wherein
the trained model includes a first trained model obtained through machine learning of a relationship between the first training image and the third training image and a second trained model obtained through machine learning of a relationship between the third training image and the second training image, and
the processor
generates, based on the input image and the first trained model, an intermediate image corresponding to an image in which the object captured in the input image is to be captured under the third imaging condition, and
outputs the prediction image based on the intermediate image and the second trained model.

7. The image processing system as defined in claim 1, wherein
the given condition includes at least one of:
a first condition relating to detection results of a position or a size of a region of interest based on the prediction image;
a second condition relating to detection results of a type of the region of interest based on the prediction image;
a third condition relating to certainty of the prediction image;
a fourth condition relating to a diagnosis scene determined based on the prediction image; and
a fifth condition relating to a part of the object captured in the input image.

8. The image processing system as defined in claim 1, wherein
the first imaging condition includes a plurality of imaging conditions under which different illumination light with different light distribution or a wavelength band is used for imaging,
the processor is capable of outputting, based on a plurality of the trained models and the input image captured using the different illumination light, a plurality of different kinds of the prediction images, and
the processor controls to change the illumination light based on the given condition.

9. The image processing system as defined in claim 1, wherein
the prediction image is an image in which given information included in the input image is enhanced.

10. The image processing system as defined in claim 1, wherein
the processor performs processing of displaying at least one of a white light image captured using white light and the prediction image, or displaying the white light image and the prediction image side by side.

11. The image processing system as defined in claim 10, wherein
the processor performs processing, based on the prediction image, of detecting a region of interest, and when the region of interest is detected, performs processing of displaying information based on the prediction image.

12. An endoscope system comprising:
an illumination device irradiating an object with illumination light;
an imaging device outputting a biological image in which the object is captured; and
a processor including hardware, wherein
the processor is configured to:
obtain, as an input image, the biological image captured under a first imaging condition and
perform processing, based on association information of an association between the biological image captured under the first imaging condition and the biological image captured under a second imaging condition that differs from the first imaging condition, of outputting a prediction image corresponding to an image in which the object captured in the input image is to be captured under the second imaging condition,
the association information is indicative of a trained model obtained through machine learning of a relationship between a first training image captured under the first imaging condition and a second training image captured under the second imaging condition,
the processor is capable of outputting, based on a plurality of the trained models and the input image, a plurality of different kinds of the prediction images, and
the processor performs processing, based on a given condition, of selecting the prediction image to be output among a plurality of the prediction images.

13. The endoscope system as defined in claim 12, wherein
the illumination device irradiates the object with white light, and
the first imaging condition corresponds to an imaging condition under which the white light is used to capture an image of the object.

14. The endoscope system as defined in claim 12, wherein
the illumination device emits first illumination light that is white light and second illumination light that differs in at least one of light distribution and a wavelength band from the first illumination light, and
the first imaging condition corresponds to an imaging condition under which the second illumination light is used to capture an image of the object.

15. The endoscope system as defined in claim 14, wherein
the illumination device irradiates the object with the first illumination light in a first imaging frame, and irradiates the object with the second illumination light in a second imaging frame that differs from the first imaging frame,
the processor performs:
processing of displaying the biological image captured in the first imaging frame; and
processing, based on the input image captured in the second imaging frame and the association information, of outputting the prediction image.

16. The endoscope system as defined in claim 14, wherein
the illumination device includes a first illumination section that emits the first illumination light and a second illumination section that emits the second illumination light,
the second illumination section is capable of emitting a plurality of illumination light that differs from each other in at least one of the light distribution and the wavelength band, and
the processor is capable of outputting, based on the plurality of illumination light, a plurality of different kinds of the prediction images.

17. An image processing method comprising:
obtaining, as an input image, a biological image captured under a first imaging condition;
obtaining association information of an association between the biological image captured under the first imaging condition and the biological image captured under a second imaging condition that differs from the first imaging condition; and
outputting, based on the input image and the association information, a prediction image corresponding to an image in which an object captured in the input image is to be captured under the second imaging condition, wherein
the association information is indicative of a trained model obtained through machine learning of a relationship between a first training image captured under the first imaging condition and a second training image captured under the second imaging condition, and
the method
is capable of outputting, based on a plurality of the trained models and the input image, a plurality of different kinds of the prediction images, and
performs processing, based on a given condition, of selecting the prediction image to be output among a plurality of the prediction images.
